(19) United States
(12) Patent Application Publication
(10) Pub. No.: US 2024/0358746 A1
(43) Pub. Date: Oct. 31, 2024

(54) METHOD OF OBTAINING STABLE SUSPENSIONS OF HETEROCRYSTALS OF TITANIUM DIOXIDE OR PARTICLES OF SILICON DIOXIDE AND STABLE SUSPENSIONS OBTAINED BY THIS METHOD FOR INITIATION OF ACTIVE FORM OF OXYGEN IN BODY AT USE IN MEDICAL FORMS

(71) Applicant: Abdula KURKAYEV, Budapest (HU)

(72) Inventor: ABDULA KURKAYEV, Budapest (HU)

(21) Appl. No.: 18/768,997

(22) Filed: Jul. 10, 2024

Related U.S. Application Data
(63) Continuation-in-part of application No. PCT/IB2022/054063, filed on May 3, 2022.

(51) Int. Cl. A61K 33/24; A61P 31/12; B01D 1/22; B01F 23/00; B01F 23/50

(52) U.S. Cl. CPC A61K 33/24; A61P 31/12; B01D 1/223; B01F 23/04; B01F 23/511; B01F 23/551; B01F 23/56

(57) ABSTRACT

The method of obtaining stable suspensions of heterocrystals of titanium dioxide and particles of silicon dioxide representing special class of quantum dots (QD). and stable suspensions obtained in such a way for initiation of active form of oxygen in the human body in use in medical forms. Starting material: Initial stuff in the form of aggregates with size more than 0.5 micrometer is mixed with an aqueous solution of pharmaceutical inorganic acid, with subsequent direction to homogenizing for the first stage of mixing, after that the obtained aqueous suspension is subjected to thermal treatment and, then aqueous suspension is directed to the rotary rotor-type evaporator periodically for evaporation of inorganic acid with suspension expense trough the rotor-type evaporator no more than 25 l/min and then the obtained activated particles are mixed with water in hydrodynamical cavitation-wave cavitational homogenizer to quasi-with regulated pulsating wave mode until obtaining stable suspension of heterocrystal of titanium dioxide or particles of silicon dioxide with size less than 450 nm, and presence on the lattice surface up to 60-80% of electronically-excited triplet oxygen ³⁺TO₂3O2 in the energy centers, namely, in the quantum dots—zones of local overheating, ensuring heat synthesis catalytic activity for formation of active forms of oxygen in the living organism human body.

The surface of Stable suspension obtained by said method is characterized by distribution of activated crystals of titanium dioxide or with size up to 1 nm being 0.3 vol %, up to 20 nm being 5-40 vol %, particles with size up to 80 nm being 10-80 vol %, particles with size up to 150 nm being 5-30 vol %, particles with size up to 250 nm being 5-20 vol %, particles with size more than 250 nm-no more than 10 vol %, and distribution of activated particles of silicon dioxide with size 40-80 nm being 10-80 vol %, particles with size 80-150 nm being 10-80 vol %, particles with size 150-250 nm being less than 30 vol %, particles with size more than 250 nm—no more than 15%. The surface of heterocrystals of titanium dioxide and particles of silicon dioxide has sorption ability, that is an important factor for use in medical forms, ensuring detoxication of an organism, elimination of hypoxia, antiviral effect of a medical agent, antipathogenous effect in the body of living organism and elimination of under oxidation processes in the human body, increasing induction of immune response of vertebrata.

### CROSS-REFERENCE TO RELATED APPLICATIONS

The present application is a continuation-in-part of and claims all rights of priority to PCT Patent Application No. PCT/IB2022/054063 for METHOD OF OBTAINING STABLE SUSPENSIONS OF HETEROCRYSTALS OF TITANIUM DIOXIDE AND PARTICLES OF SILICON DIOXIDE AND STABLE SUSPENSIONS OBTAINED BY THIS METHOD FOR INITIATION OF ACTIVE FORM OF OXYGEN IN BODY AT USE IN MEDICAL FORMS, designating the United States of America, filed on May 3, 2022 (pending).

### TECHNICAL FIELD

The present invention relates to the field of obtaining stable suspensions of nano-size particles of heterocrystal minerals and the methods of their obtaining.

Industry is highly interested in suspensions of titanium dioxide and silicon dioxide nanoparticles because of diversity of their properties and a great number of applications where they can be used.

### BACKGROUND OF THE INVENTION

Preferably for nanoparticles preparation minerals with heterocrystal structure are used, especially rutile (TiO₂; (Ti, Nb, Fe) O₂), anatase (TiO₂, (Ti, Nb, Fe) O₂) and quartzite (SiO₂). These minerals are widely extended in the nature and could be easily dispersed.

Rutile is a natural mineral, being a principal titanic ore as a metal source, used in high-technology alloys thanks to its low density, high strength and corrosive resistance. Microscopical inclusions of rutile could be found in quartz, tourmaline, ruby and sapphire. This stable compound forms in the process of formation at high temperatures and pressures in form n(SiO₂)-n(TiO₂), but at lowering of temperature and decrease of pressure these two components separate forming crystals of rutile captured by the quartz crystals.

In particular, because of their physico-chemical characteristics nanoparticles become highly useful in different fields: treatment of different diseases, for example, infectious, bacterial or viral infections, cancer therapy, wounds healing, anemia treatment, etc.

WO2007048634 «NANOPARTICLES OF HETEROCRYSTAL FOR USE AS A MEDICAL AGENT AND METHOD OF THEIR OBTAINING».

Synthesis optimized at low concentration is hard to repeat at higher concentration without problems in stability and aggregation, that is why it is necessary to emphasize that difficulties arising in industrial adaptation are in synthesis of systems stable in time with high concentration and controlled particle sizes.

From the technical area the patent EP2462915A1 is known Publication 24.12.2014 Priority PCT/JP2010/062958 from 2010 Jul. 30. «Organic-inorganic composite particles and process for production thereof, dispersion containing the particles, and cosmetic containing the particles».

Organo-inorganic composite particles for cosmetics, including particles of inorganic oxide, each having cationic charge on the particle surface, and molecules of polymeric gel, obtained of a natural substance and having anionic functional group and one or several hydroxyl groups in a molecule, at that said molecules of polymeric gel are electrostatically bound with surfaces of inorganic oxide particles. Inorganic oxide particles are the particles of oxide or complex oxide of at least one metallic element, chosen from cesium, magnesium, calcium, barium, cerium, titanium, zirconium, vanadium, iron, zinc, aluminium and silicon (except for silicon oxide particles).

Average diameter of organic-inorganic composite particles desirable fall in the range from 0.1 to 300 mcm, preferably from 0.15 to 280 mcm. But the particles of organic-inorganic composite could be also used, having average diameter of particles less than 0.1 mcm, a force of dispersing particles increases, that complicates particles treatment, therefore such particles are undesirable. If the average particles diameter exceeds 300 mcm, the force of spontaneous deposition increases, that makes difficult to prepare homogenous dispersion of particles in a dispersion medium. At that at the final stage after filtration and drying of organic-inorganic composite particles water is added for final dispersion.

For a cosmetic agent the characteristics of homogenous flowability of the obtained suspension is given.

From the technical field the European patent No. 016541WO 2007/118884 2007.10.25 (PCT PCT/EP2007/053761) is known, «Compositions of magnetic nanoparticles and their use».

The invention relates to use of biocompatible nanoparticle or nanoparticles aggregate in combination with external non-oscillating magnetic field, where said nanoparticle includes: a) a core containing magnetic material; b) biocompatible cover surrounding the core; and, optionally, c) a marking agent, where the external diameter of the cover is less than about 100 nm, for preparation of composition, where said composition does not contain any other means for targeting on the cell. The present invention also relates to obtained compositions and their use in the healthcare area, for cancer treatment or in diagnostics (for example, visualization), for monitoring of the tumor development.

Ferromagnetic material is chosen from the group consisting of iron, nickel, cobalt, gadolinium, samarium, neodymium, boron, aluminum and any mixture of them. Material of ferromagnetic core is in form of oxide, hydroxide or metal.

Compositions may exist in solid or liquid form (suspended nanoparticles), for example, in the form of paste or aerosol. «The method of obtaining multicomponent particles» is known according to the patent US 2010/0262115 including the stage of obtaining inorganic nanoparticles. Said invention is destined for an agent containing nanoparticles, for cancer treatment, pharmaceutical compositions containing them, and the methods of their use for medicines delivery and cancer treatment by ultrasound or light.

Nanoparticle containing internal volume containing derivative of hypocrelline-B and polyvinylpyrrolidone cover, encapsulating internal volume of hypocrelline-B is SL052.

Nanoparticle is bound with a marking agent being detected. Nanoparticle where the detected marking agent is chosen of fluorescent or other light-emitting marker, radioactive indicator or contrasting agent. Nanoparticle is used in combination with one or more pharmaceutically admissible carriers for formation of pharmaceutical composition for tumors treatment.

Polyvinylpyrrolidone (PVP) (having average molecular mass of 40 000 Da) and a derivative of hypocrelline-B (designated as «SL052») in a method of deposition was used for obtaining SL052-NPS. 1.5 ml 0.5% (7.5 mg/ml) of PVP water solution were added to 6 ml of water when mixing at the room temperature. Ten minutes later 1.59 ml 4.6 mm SL052 in dimethyl sulfoxide (DMSO, Fisher Scientific) were added to this mixture. The obtained solution was mixed during ten minutes in the dark in order to obtain nanodispersion with nanoparticles having size of 136 nm.

Suspensions reported in the literature are obtained either immediately in the course of synthesis or by the way of synthesis and deposition of nanopowder with subsequent redispersion in a solvent in presence of additives [P K Khanna et al. Mater. Lett. 61 (2007) 3366]; however, it is necessary to note that for improvement of the system stability in time, for facilitation of scaling and in order to avoid the stage of extraction, washing out and repeated dispersion of powder nanoparticles are preferably converted in suspension immediately after synthesis.

The methods of obtaining stable suspensions of metallic nanoparticles Au, Ag, Cu, Pd, Pt, Fe are known, realized in aqueous medium at low temperature, under pressure and in atmospheric environment at heating using microwave apparatus, as well as stable suspensions of nanoparticles obtained in such a way. Publication of the application is PCT/EP2010/052534WO 2010/100107 (10.09.2010).

The term «metallic nanoparticles» designates particles of Au, Ag, Cu, Pd, Pt, Fe with sizes in the range from 1 up to 250 nm, preferably from 1 to 100 nm; in particular, the invention relates to nanometric suspensions of Ag and Au.

The elaborated method consists of adding saline precursor of metal to reactionary medium, already heated up to the reaction temperature and containing reducing agent, chelating agent and a catalyst in strictly determined molar ratios of chelating agent/metal, reducing agent/metal and catalyst/metal.

The method ensures obtaining of colloidal suspensions having high concentration and being stable in time (during time periods more than 7 months).

Suspensions obtained in such a way are characterized by the rate of average dynamic light scattering (the DLS method) about 20-30 nm and show monodisperse distribution before concentrations, and for higher concentrations bimodal distributions are noted in presence of the basic particles population in the size range of 5-10 nm.

Use of suspensions of metallic nanoparticles of Au, Ag, Cu, Pd, Pt, Fe is restricted in view of complexity of withdrawal from the human body because of unstable suspension condition. In connection with particles agglomeration setting of sediment occurs, and it is not isolated/sampled from organism.

Au is chemically inert, does not interact with acids, aqueous medium of organism and oxygen and practically is not isolated/sampled from organism.

A study of biological properties of nanoparticles of silver has shown that in aqueous medium these particles are essentially in the oxide form. Silver oxide has positive biological activity (antimicrobial and antitumoral) in particular, owing to the mechanism of photocatalysis of passage of dissolved atmospheric oxygen PO₂ in the active form of oxygen (AFO).

Moreover, silver in suspension has tendency to aggregation, loss of oxide form (passage in catalytically inactive quasi-metal particles), that is negatively estimated by the toxicologists.

The technical problem for solving of which the present invention is intended, is obtaining stable suspensions on the base of crystals, bio-dissociable, potentially applied in medical forms composition, namely, two products of titanium dioxide or silicon dioxide, acquiring expressed biological activity in the case of their special structure at obtaining suspensions from them, possibility of the crystals surface to enter into chemical-physical bonds with the energy reaction centers.

The technical result is obtaining of long-time stability suspensions of heterocrystals of titanium dioxide or particles silicon dioxide characterized by presence of oxygen on their surface, ensuring initiation of an active form of oxygen (AFO) in the human body.

The product representing heterocrystals of titanium dioxide is mentioned hereinafter as activated titanium dioxide. The internal part of particles of activated (nanocrystalline) titanium dioxide still has crystalline structure of pigmental titanium dioxide, used as excipient in pharmaceutical compositions. Improvement of interphase properties (physical, technological and catalytic characteristics) due to activation of nanoparticles do not result in formation of a new chemical compound, but to the same compound with changed colloidal and surface properties, namely, presence of oxygen in the structure, high absolute value of Zeta-potential on the crystal surface, ensuring the initiation of AFO in a live organism.

The chemical precondition of the reaction of activation of heterocrystals of titanium dioxide or particles of silicon dioxide is modulation of electrostatic properties of the surface (increase of Zeta-potential due to increase of the surface charge density) due to chemisorption of H⁺ and formation of photocatalytic domains (lattice imperfections) due to aggressive acid treatment of the surface.

The ratio of particles and crystals charge is one of the factors determining physical stability of suspensions.

By determining Zeta potential in preparation of suspension it is possible to quantify the magnitude of electrostatic repulsion between particles and so the higher is the zeta potential, the higher is the physical stability. Usually, the particle charge is defined in terms of quantity by Zeta-potential, being measured, for example, using electrophoretic mobility of particles in the electric field.

### SUMMARY OF THE INVENTION

The method of obtaining stable suspensions of heterocrystal of titanium dioxide and particles of silicon dioxide is characterized by the fact that the starting material is in the form of aggregates with size more than 0.5 micrometer is mixed with an aqueous solution of inorganic acid, with subsequent direction to homogenizing for the first stage of mixing, after that the obtained aqueous suspension is subjected to thermal treatment, then aqueous suspension is directed to the rotary evaporator working under pressure lower than 100 kPa at temperature no more than 70° C. for evaporation of inorganic acid and then the obtained activated particles are mixed with water in hydrodynamical cavitational homogenizer with regulated pulsating wave mode until obtaining stable suspension of heterocrystal of titanium dioxide or particles of silicon dioxide with size less than 450 nm, and presence on the lattice surface from 60 to 80% of electronically-excited third-stage triplet oxygen (³TO₂) in the energy reaction centers, namely, in the quantum dots (QD)—zones of local overheating, ensuring catalytic activity for formation of Active forms of oxygen (AFO) in the living organism.

For obtaining stable suspensions of heterocrystals TiO₂ as starting material, the powder TiO₂ is used, containing modification of anatase and rutile in ratio preferably from 10:90 up to 90:10, more preferably from 60:40 up to 40:60.

Initial Starting material is correspondingly mixed with pharmaceutically approved, for example, hydrochloric or sulfuric acid in the form of 0.0001 N-0.1 N aqueous solution.

At the expense of using aggressive acid treatment the level of microbial, pyrogenic and other hydrolyzable organic contaminations decrease, that is highly important for subsequent elimination of toxicity of obtained stable suspensions and their application in medical agents composition.

Pharmaceutically approved acids may be also chosen from the following acid range: mainly hydrochloric acid, sulfuric acid, as well as metaphosphoric acid, methanesulfonic acid, nitric acid, phosphoric acid, sulfuric acid, tartaric acid.

Stability of obtained suspensions, in particular, is significantly influenced by pH of a medium obtained as a result of acid treatment of starting material.

Stability of obtained suspensions is ensured by low or neutral value of the pH of the suspension medium.

The obtained mixture is directed to the primary stage of mixing into the homogenizer, being, for example, an apparatus provided with supplying nozzle, that trough the ejection channels is interconnected with coaxial auxiliary chamber, attached to it by a vortical chamber and a chamber of mixing, connected with the discharging nozzle for output of homogenous aqueous dispersion after the first stage of mixing.

Then obtained after homogenizing aqueous suspension of heterocrystals of titanium dioxide or particles of silicon dioxide is directed to the ultrasonic bath with ultrasonic frequency of 20-90 kHz, where it is kept at the temperature no more than 70° C. during not more than 2 hours.

Dispersive medium is evaporated in the vacuum rotary evaporator under pressure lower than 100 kPa at the temperature not more than 70° C. for evaporation of pharmaceutically approved acid.

During evaporation of a dispersion medium volatile acid vapors are released. At this time the surface of particles and crystals is activated. After that activated powder TiO₂ or SiO₂ is obtained, having developed surface and special structure of crystals and particles.

A powder of activated TiO₂ or SiO₂ particles is directed into the hydrodynamical cavitational homogenizer, where water is added in doses for obtaining from 0.0001%-10% of stable suspension of TiO₂ heterocrystals or SiO₂ particles.

The process of treatment in the hydrodynamical cavitational homogenizer lasts from 10 minutes depending on specified indices required for suspension.

The homogenizer contains the blocks for treating fluid medium sequentially connected with one another along the flow of mixed medium, at that the first block of preliminary mixing contains the inlet nozzle of basic medium, the inlet nozzle of admixed medium and the outlet channel of mixed medium, through which the last one overflows in the homogenization block and then overflows in the block of controlled output of homogenized product, at that, according to the invention, in the homogenization block the cavitational mixing chamber is made with the reflector of flow mix at the output of the chamber, fulfilled in the form of narrowing along the flow chamber walls, at that in the chamber the insertion with the central channel is installed, on the external surface of the insertion the spiral groove is fulfilled, the insertion contacts with the internal wall of the chamber with possibility of forming the open-end spiral channel along the spiral groove and mixing of flows of spiral and central channels in the cavitational chamber before the reflector, and the central channel is provided with narrowing and widening sections, at that the block of the controlled output is provided with the controlling rod of the flow medium reflector, the projection of which is located in the communication channel with the outlet of the homogenizing block, and the controlling rod is connected with the means of changing location of its projection in the communication channel.

In the cavitational chamber the insertion with the central channel is installed, on the external surface of the insertion the spiral groove is fulfilled. The insertion contacts with the internal wall of the chamber with possibility of forming the open-end spiral channel along the spiral groove and mixing of flows of spiral and central channels in the cavitational chamber before the regulated reflector.

A fluid product fed to the chamber is twisted in the spiral channel, formed by the spiral groove, and forms pulsating intensive vortical layer.

In the homogenizing chamber the intensive vortical layer is formed on the flow surface, that at hitting against the reflector excites turbulent wave pulsations considering thermophysical influence of hitting, where temperature of treated fluid medium/product for one processing circuit rises from 10%.

The structure of the cavitational homogenizer ensures high degree of homogenizing the obtained suspension.

The suspension stability is determined by presence of procedures and operations of the method, use of starting material, namely, combination of anatase and rutile in obtaining suspension of titanium dioxide heterocrystals, use of an acid solution, application of different types homogenizers at the first stage of mixing and hydrodynamical cavitational homogenizer for final mixing and obtaining stable suspensions.

The design of the cavitation homogeniser provides a high degree of homogenisation of the resulting slurry. (PCT Application No. PCT/IB 2022/054057 dated 3 May 2022 International Publication WO2023/214200).

Stable suspension of TiO₂ crystals or SiO₂ particles obtained in accordance with the developed method, ensures the content of a structure of crystals and oxygen particles on the lattice surface in a metastable electronically excited third triplet state of (³⁺TO₂), while TiO₂ crystals have a zeta potential of +30-+15 mV, and SiO₂ particles have a zeta potential −20-−15 mV, TiO₂ crystals or SiO₂ particles are characterized by the presence of sorption properties.

Zeta potential is measured by Malvern Zetasizer Nano ZS instrument (Phase Analysis Light Scattering method). The range of Zeta-potential is 10-100 mV.

Obtained stable suspension is characterized by distribution of activated titanium dioxide crystals with size to 1 nm, that is 0.3 vol %, to 20 nm is 5-40 vol %, particles with size to 80 nm are 10-80 vol %, particles with size to 150 nm are 5-30 vol %, particles with size up to 250 nm are 5-20 vol %, particles with size more than 250 nm are not more than 10 vol %, and distribution of activated silicon dioxide particles with size 40-80 nm are 10-80 vol %, particles with size 80-150 nm are 10-80 vol %, particles with size 150-250 nm are less than 30 vol %, particles with size more than 250 nm are not more than 15 vol % on the base of the data obtained in the analysis with the photon correlation spectroscopy instrument Malvern Zetasizer Nano ZS and is characterized by presence on the lattice surface from 60 to 80% of electronically-excited third-stage triplet oxygen (³⁺TO₂) in the energy centers, namely, in the quantum dots-zones of local overheating, ensuring catalytic activity for formation of active forms of oxygen in the living organism.

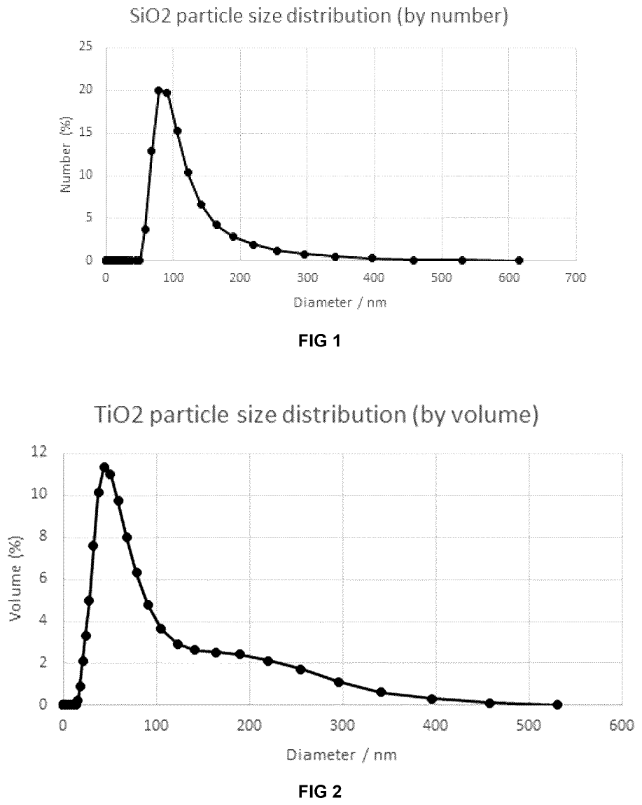

The FIG. 1 shows distribution of silicon dioxide particles with different sizes in suspension by volume.

The FIG. 2 shows distribution of titanium dioxide particles with different sizes in suspension by volume.

Polydispersity of obtained suspensions determines their unique properties at forming medical agents, that ensures not only targeted delivery into the area of pathological processes, but also unique mechanisms of action for use in the medical agents.

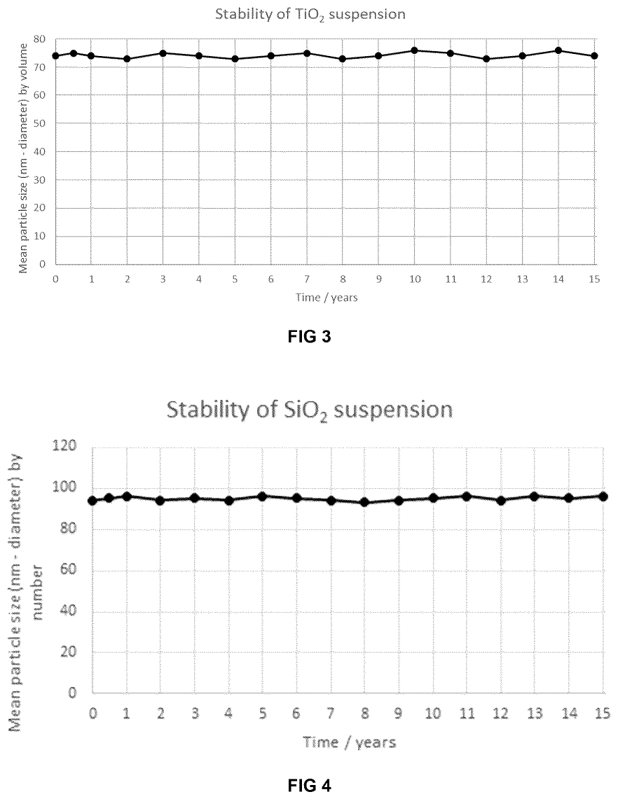

Stability in time for suspension obtained in accordance with the elaborated method is from 24 months and during no less than 15 years, in the tests for stability no changes are noted in optical properties (spectroscopy in UV visible range, FIG. 3, FIG. 4) and in the particles size (DLS), that indicates absence of changes in quantity and sizes of particles in the course of time.

Hereinafter the tests of stability examination for the QD series suspension ADAM QD/T (TiO₂) are shown.

The Tables 1 and 2 show Results obtained during normal (long term) stability study (+25±1° C.)

The obtained data showed that: pH of these samples did not show significant tendentious alterations. Dynamic viscosity of these samples did not change significantly upon storage. The colloidal stability of the product was found outstanding. No tendentious changes were found in the particle size of the product. The titanium dioxide content of the product did not change during storage. Sterility and endotoxin content investigations were only prescribed to be performed at the end of the storage study. These samples were found sterile and containing lower concentration of endotoxin than specified.

In accelerated, and also long-time examinations of stability Appearance of the samples stored at different storage conditions did not show alterations from the specification.

The Tables 3 and 4 show Results obtained during normal (long term) stability study (QD) ADAM QD/S (SiO₂), T=+25±1° C.

The obtained data showed that: pH of these samples did not show significant tendentious alterations. Dynamic viscosity of these samples did not change significantly upon storage. The colloidal stability of the product was found outstanding. No tendentious changes were found in the particle size of the product. The silicon dioxide content of the product did not change during storage. Sterility and endotoxin content investigations were only prescribed to be performed at the end of the period planned for the storage study and found to meet the product specifications.

In accelerated, and also long-time examinations of stability Appearance of the samples stored at different storage conditions did not show alterations from the specification. Thereby, continuous stability of suspensions is ensured as a result of a complex of procedures and operations of the elaborated method, in particular due to use of different modes of the special cavitation-waved homogenizers at the process stages.

Presence on the crystal surface of oxygen O₂, occupying from 60 to 80% of structure and presence in the ruptures of the developed surface of crystals lattices of a great number of ionic groups of ligands ensures formation of excitonic structures, ionic bonds and zones of local energy overheating, i.e. the quantum dots, where O₂ is in metastable electronically-excited third-stage triplet condition (³⁺TO₂) with unique characteristics of conversion into biological activity—singlet condition (S¹⁻³O₂).

Illustration of measuring quantity of electronically-excited triplet oxygen (³⁺TO₂), on particles surface was executed as follows. Researchers performed experiments on a rabbit, male with 0.75 kg weight (without injuries). Through the needle 5 ml of suspension was injected, where 5 ml is injection water and 3 mg TiO₂, at the entry inside the needle opening there was a light guide for feeding laser photons and supplying injection suspension simultaneously. The photonic-thermic procedure was executed with the laser IR 960 nm with 3 Wt power, dose 20 J regulating synchronously.

In the local treated part of the examined rabbit, with a help of an additional needle the sensor for measuring O₂ was installed, and the analyser of automatic chemiluminiscent gas analyser of singlet oxygen S¹⁻³O₂ (the model FOMS-200 OXYGEN, made in FRG). Presence of conversion of ³⁺TO₂ triplet oxygen with presence in oxygen structure in metastable electronically-excited condition into singlet condition (S¹⁻³O₂) was fixed.

Based on experimental data, an example of calculating the amount of O₂ oxygen on the crystal surface, which occupies from 60 to 80% of the structure, is given in Example 4. The surface of TiO₂ crystals or SiO₂ particles has sorption ability, that is an important factor for use in the medical forms.

Active chemical groups in the places of ruptures of crystal lattice easily form the chemical bonds with the same active molecules of environment.

The existing mechanisms of sorption interaction between different substances molecules are:
A—non-specific binding by porous structures;
B—same when the sorbate is a fibrous material;
C—ionic sorption;
D—covalent;
(all types of sorption are possible).

As a result of the fact that the surface of TiO₂ crystals or SiO₂ particles, containing from 60 to 80% of oxygen, and presence in the ruptures of developed lattices surface of a great number of ionic groups of ligands, formation of excitonic structures, ionic bonds and a range of zones of local energy overheating is ensured, all the types of sorption are possible, that determines detoxication possibilities of the obtained stable suspensions.

The researchers showed, that in one dose of the preparation 1 mg/ml including stable suspension of TiO₂ crystals or SiO₂ particles, their sorption capacity as a result of said sorption properties of crystals and particles is two times higher in comparison with the existing preparations.

The studies of sorption properties of aqueous suspensions of TiO₂ and SiO₂ were carried out.

Comparison as a means of detoxification (efferent therapy)—Cyclolab Kft, study protocol No 3.Sorbent. The aim of the study is to demonstrate the products in a comparison of detoxification capabilities, where TiO₂ and SiO₂ products form chemical bonds with metabolic waste molecules (by-products) on the active sites of crystal and particle lattices: Studies were carried out in a rabbit model, 3 kg, comparing the sorption efficiency of exogenous creatinine:

1—Untreated control group (no exposure either by creatinine or suspension)
2—10 grams carbosorbent, 50 ml intravenous perfusion during 20 minutes of administration
3—TiO₂ (ADAM-QD/T) 1 ml/0.6 mg*
4—SiO₂ (ADAM-QD/S) 1 ml/0.6 mg*
   *—i.e., 0.2 mg/l kg body weight, intramuscular injection
5—Creatinine exposed model/study group untreated by suspension

Table 5 summarises the exogenous creatinine values in the experiment: (the result is at least 3.5 times the upper limit of the normal range: 0.5-2.5 mg/dL).

Conclusion:—Based on this study, aqueous suspensions of TiO₂ and SiO₂ crystals or particles were found to be effective creatinine sorbents in vivo. This mechanism allows substances acting on a living organism to be considered as having a therapeutic effect aimed at removing harmful, toxic or metabolic waste compounds from exogenous source or originating via endogenous manner.

The studied nanoparticles provide safe complexes of formulations with other active components, due to their binding on their surfaces of crystals and particles, as well as the ability to target delivery to pathogenic areas, then adsorb toxins and remove them from the body.

The obtained polydisperse suspension has biological activity due to not only photocatalytic properties, but also acquire the properties of thermocatalysis, which allows for the synthesis of ROS in the body of activation, for the first time achieved by exertion of the human body temperature of 36.6° C.

Every neutral inorganic object found in a living tissue, inevitably gets in the category of the “foreign bodies”, i.e. relates to formations with principally pathological character.

Biocompatible TiO₂ crystals (ADAM-QD/T) and SiO₂ particles (ADAM-QD/S) are the only most indifferent in use in different forms of medical agents. Absence of general and local toxicity is confirmed by the results of toxicological researches. The side effects in examinations of toxicity and examination of the repeated dosed were not revealed, and the results of researchers also confirm absence of mutagenicity and elastogenicity. Parenteral tolerated dose for TiO₂ is 21.72 mg/kg bw, and for SiO₂ 11.39 mg/kg bw. Oral tolerated dose for TiO₂ is 2172 mg/kg bw, and for SiO₂ 1139 mg/kg bw (Dossier No. 488.729.2117; 488.729.2119 from 06.07.2010, TOXI COOP Zrt).

As an integral part of the safety profile investigations a wide scope of preclinical OECD (The Organization for Economic Cooperation and Development) conforming tests were conducted to reveal the practical applicability (i.e. in clinical use) of the test substances according to present invention in various therapeutic areas. A series of in vitro and in vivo GLP toxicological studies were performed to obtain detailed analytics on the safety and kinetics of TiO₂ and SiO₂ products. In 28-day toxicological studies in rats and dogs, TiO₂ and SiO₂ products demonstrated a complete absence of toxicological effects and pathological conditions in males and females when administered intramuscularly. The study included a toxicokinetic assay, evaluation of safety pharmacology, evaluation of local tolerance, a 28-day experimental phase followed by a 28-day recovery phase (Dossier #488-407-7122, #488-407-7234, #17/2/L, #18/22/L).

In Studies #488-827-7578 for TiO₂ and #488-827-7579 for SiO₂, correlation (pharmacokinetic) parameters were also considered for multiple administration for subcutaneous, oral, vaginal, rectal and topical routes of administration. The results of these studies demonstrate the kinetics for the determination of TiO₂ and SiO₂ levels at different routes of administration. Table 6 displays the bioanalytical results for TiO₂ (ADAM-QD/T) product.

The results of the repeated intramuscular toxicity study with toxicokinetic sampling and recovery in Beagle dogs conducted under GLP (Good Laboratory Practice) conditions are presented in Table 6. The aim of the study was to determine the safety of the intramuscularly administered tested formulation containing TiO₂ (ADAM-QD/T).

The study included toxicokinetic sampling, safety pharmacology assessment, local tolerance evaluation, and a 28-day experimental phase followed by a 28-day recovery phase. Based on the results obtained from the study, it can be confirmed that the tested product did not induce pathological conditions in the experimental animals, thus it is safe. Additionally, analytical investigation was carried out on plasma samples and various organ homogenates by ICP-MS subsequent to chemical digestion of the organic matrix and quantitative dissolution of TiO₂ (ADAM-QD/T). The results showed that the test substance after administration can be well detected and quantified in plasma and different tissues, whereas the organ distribution of the product is uniform.

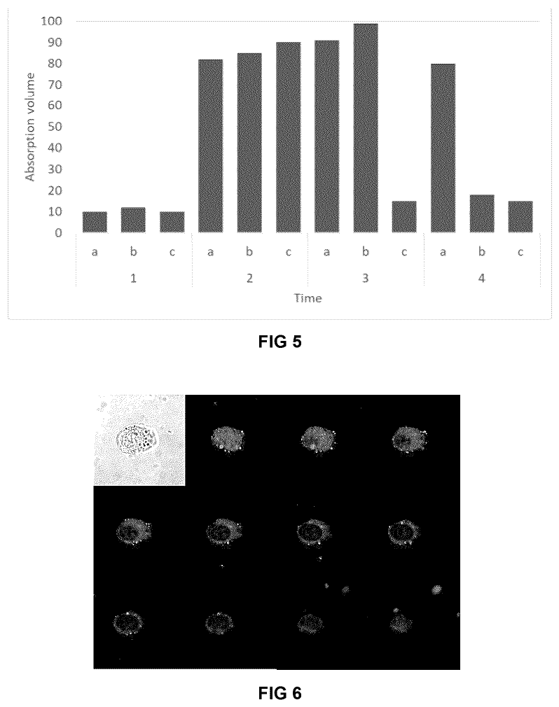

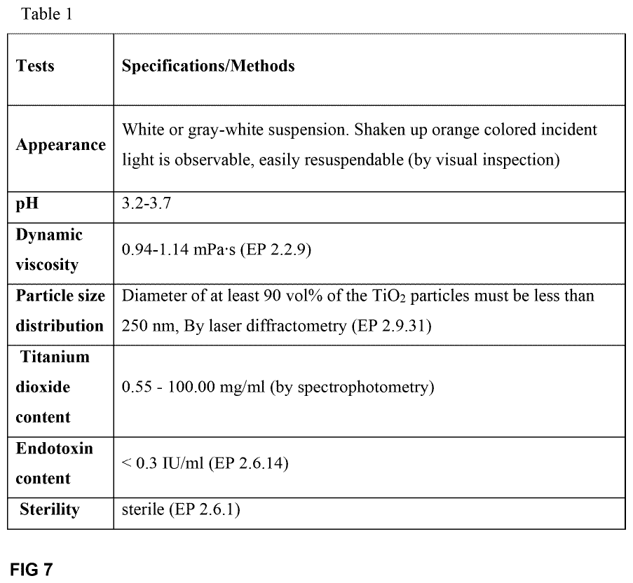

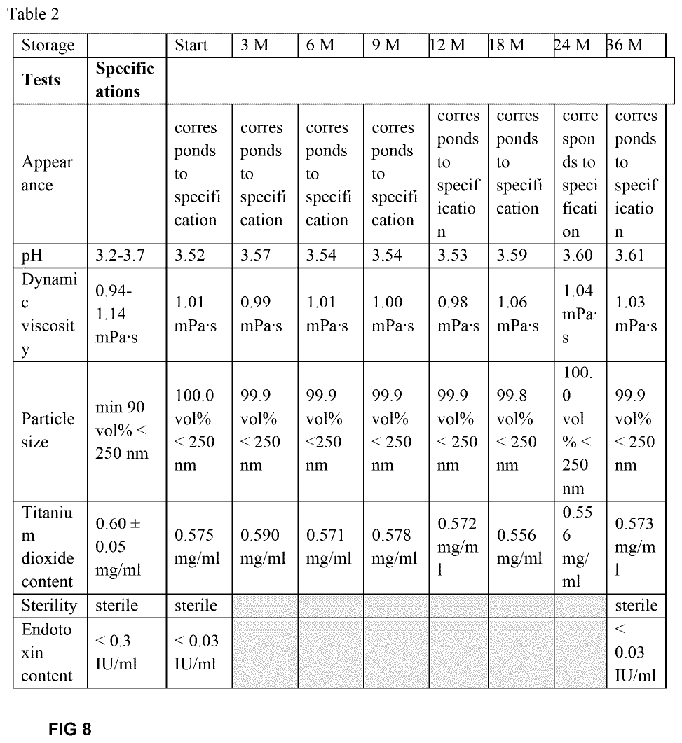

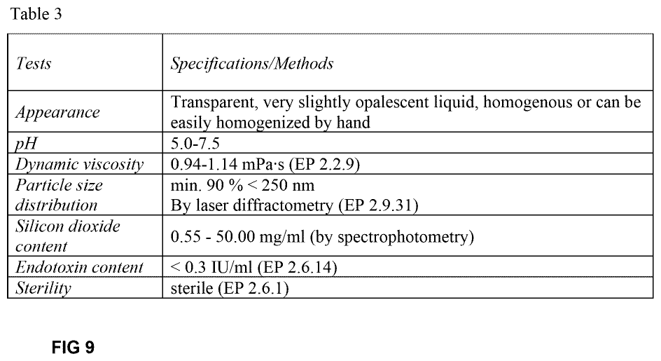

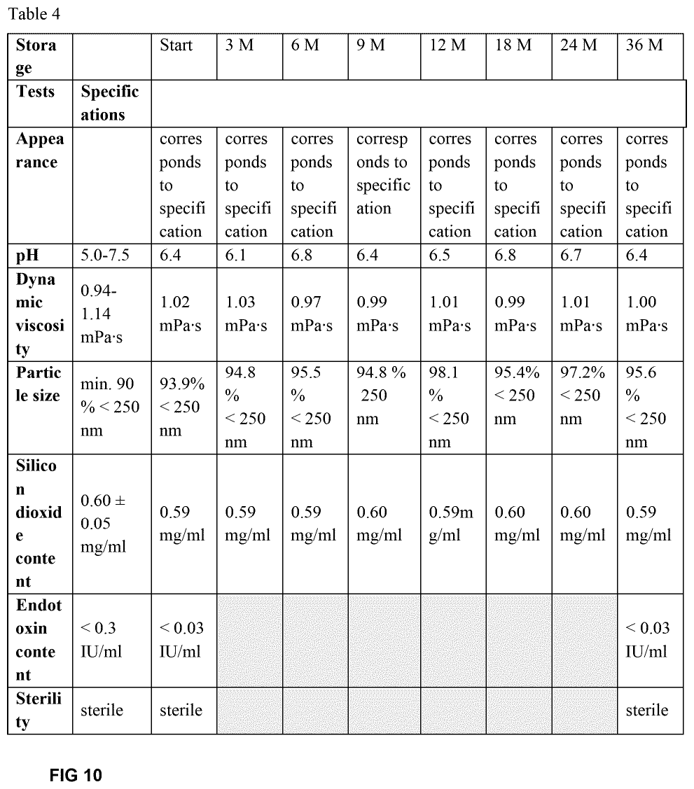

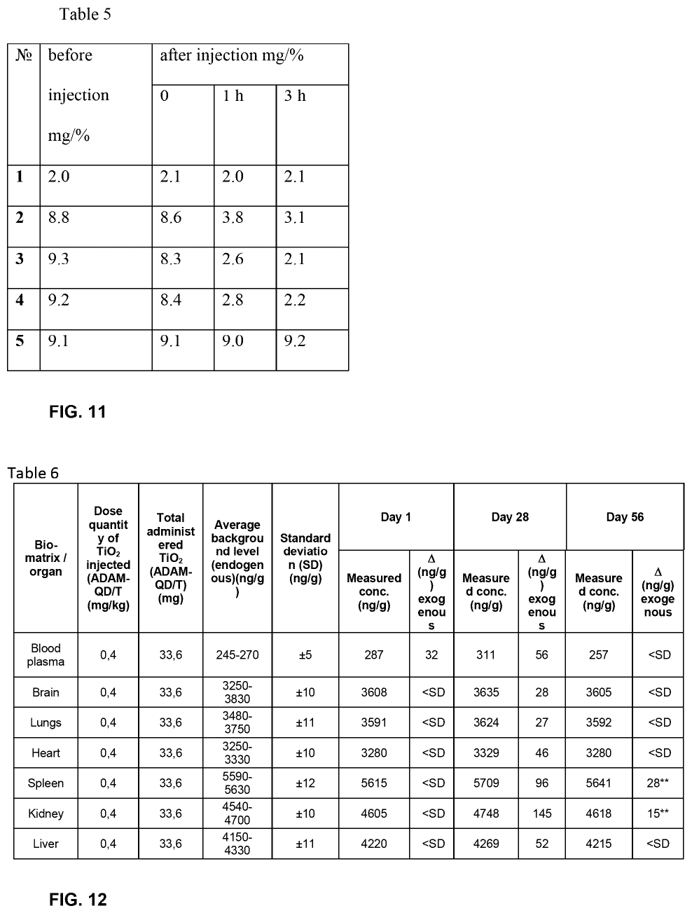

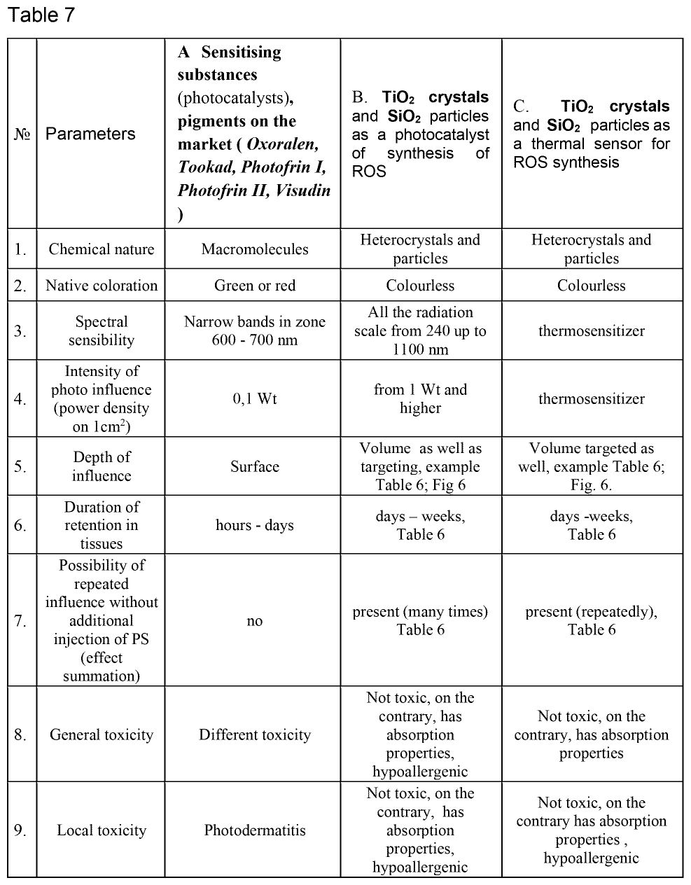

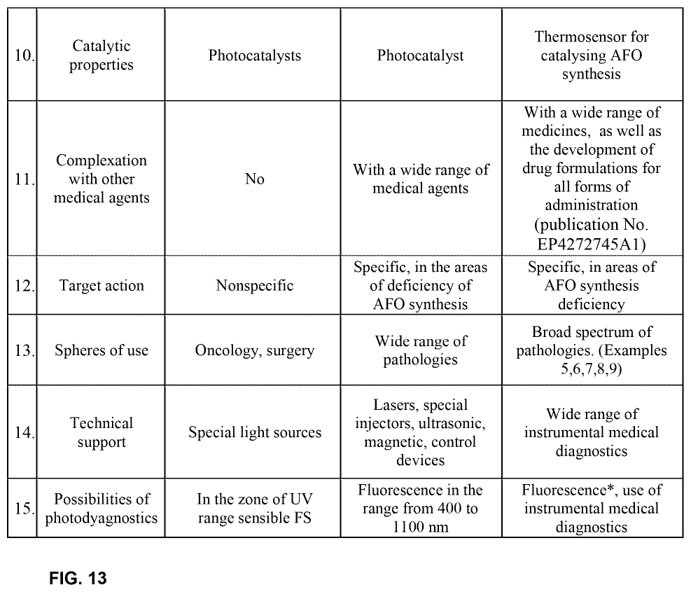

Blood plasma before TiO₂ (ADAM-QD/T) administration contained 255±5 ng/g Ti as endogenous (background) level. This concentration was recorded representing the background concentration without any exposure. On day 1 after administration, the plasma level elevated to 287 ng/g indicating that the recorded additional 32 ng/g TiO₂ (ADAM-QD/T) concentration was due to the external exposure (denoted by A). On day 28, the TiO₂ concentration further increased to 311 ng/g establishing a Δ=56 ng/ml peak in the exogenous TiO₂ (ADAM-QD/T) increment compared to the background value. On day 56, the level of TiO₂ decreased to the background concentration (A becomes insignificant, i.e. the difference is within experimental error compared to the initial value) showing total elimination from the studied biomatrix. Very similar tendencies in the kinetic curves were obtained for the organs, some residual trace amounts remained however in spleen and kidney (by extrapolation, their elimination was delayed by several weeks) which is confirmed by Table 6, and also in FIG. 6. FIG. 6 shows the two-way transmembrane trafficking of the nanoparticles visualized by a fluorescent dye (rhodamine) adsorbed on the surface of the crystals under a confocal microscope evidencing the uptake and release of the nanoparticles to/from a mammalian (mouse fibroblast) cell. The mechanism of cellular uptake is endocytosis, whereas release is performed by exocytosis.

The concentrations of endogenous titanium and silicon were determined by measuring samples from untreated animals. The biological matrices studied, including organ tissues, contain varying levels of endogenous Ti and Si ranging from 200-6000 ng/g. Titanium dioxide and silicon dioxide are common compounds in the body (by mostly food intake). Such abundance constantly influences the inevitable proliferation of endogenous levels of titanium and silicon. Their impact and accumulation are inevitable, for example in experimental animals level fluctuations are registered upon contact even with bedding, cages and other factors.

The exogenous exposure to TiO₂ (ADAM-QD/T) used in the study, as shown in Table 6, shows that the minor therapeutic contribution of TiO₂ (ADAM-QD/T), compared even to the fluctuations in endogenous concentrations due to the above factors during the study period, can be attributed to the fact that the intentional exposure did not lead to serious imbalance of endogenous concentrations. A similar trend was found in SiO₂ (ADAM-QD/S) studies.

The investigation was carried out in order to establish suitable preclinical background prior to human safety studies: such findings provide essential and adequate grounds for further clinical investigations and human applications of the product. In Table 6, Δ ng/g means the concentration elevation within the biomatrix that is specifically due to exogenous (external) exposure of TiO₂ due to intramuscular administration of the formulation; and **-excreted between 9 and 11 weeks. The data collected in Table 6 not only show that nanosized TiO₂ (ADAM-QD/T) is distributed nearly evenly in the body of the test animals, but also that administered TiO₂ is being eliminated from the organs within a few weeks.

A similar study was conducted with nano-sized SiO₂, which showed similar pharmacokinetic properties to nano-sized TiO₂ (ADAM-QD/T), indicating that the administered SiO₂ (ADAM-QD/S) was distributed essentially evenly throughout the target organs of the tested animals, and that the administered SiO₂ left these organs quantitatively relatively faster in comparison with TiO₂.

It is important to note that the results of the preceding 28-DAY INTRAMUSCULAR TOXICITY STUDY INCLUDING TOXICOKINETICS AND SAFETY PHARMACOLOGY TESTING OF TiO₂ (ADAM-QD/T) and SiO₂ (ADAM-QD/S) IN RATS align with those of the Dog research, namely that the products were eliminated from the organs by the end of the study. This consistency provides further confirmation of the safety of the tested formulation and aids in the progression of clinical investigations.

The reverse bacterial mutation study under the applied experimental conditions showed that the investigated products TiO₂ and SiO₂ do not cause gene mutations in the genome of Salmonella typhimurium strains TA98, TA100, TA1535, TA1537 and Escherichia coli WP2 uvrA. (Dossiers #488-471-7227 and #488-471-7230).

The in vitro mammalian chromosomal abberations test did not induce chromosomal abberations in Chinese hamster V79 cells when cytotoxic concentrations of 250-1000 μg/ml of TiO₂ and SiO₂ products were tested. Based on the results, the TiO₂ and SiO₂ products can be considered non-clastogenic, which means that the presence of TiO₂ and SiO₂ in the studied concentrations does lead to any harm in DNA strands, therefore these products are not carcinogenic. (Dossier #488-473-7228 and #488-473-7231).

Further confirmation of the safety of TiO₂ and SiO₂ products was obtained from the mouse micronucleus test, in which groups of mice treated with TiO₂ and SiO₂ products demonstrated no biologically and statistically significant increase in the incidence of MPCE compared to the negative and control groups (Dossier #488-474-7229 and #488-474-7232).

Thus, a series of toxicological studies have shown that stable suspensions of TiO₂ and SiO₂ are safe (i.e., they do not cause organ dysfunction and do not harm genetics), which is confirmed by the data given in Table 6 and examples 5,6,7,8. Conducting these studies in accordance with OECD requirements facilitates the transition to the clinical trial phases.

Other metals, such as iron metal compounds Fe automatically get in the forbidden area for use in pharmaceutics and obtaining pharmaceutical receipts of preparation. Metals of iron Fe are of the “heavy” group, are considered «xenobiotic», and also are not released from the body of living organism.

A certain exception is a group of precious metals. But in fact only silver is regarded in this group. Examination of biological properties of silver nanoparticles showed, that in aqueous medium (such is it in the organism tissues) these particles are essentially in the oxide form. Silver oxide has positive biological activity (antimicrobial, antitumoral), in particular, owing to the mechanism of photocatalysis of conversion of dissolved atmospheric oxygen (PO₂) into active form of oxygen (AFO). However, silver has a number of imperfections: aggregation arises in suspension, losses of oxide form (conversion into catalytically inactive particles of quasimetal).

Biological inertness of TiO₂ crystals and SiO₂ particles is well-known, and expressed and regulated biological activity they acquire in the course of a special method of their obtaining, as a result of which the local zones of energy overheating appear (de la Hoz A, Díaz-Ortiz A, Moreno A. Microwaves in organic synthesis. Thermal and non-thermal microwave effects. Chem Soc Rev. 2005 February; 34 (2): 164-78. doi: 10.1039/b411438h. Epub 2005 Jan. 12. PMID: 15672180), ensuring catalytic activity for formation of active forms of oxygen in the body of living organism.

Under normal conditions atmospheric oxygen is in its ground state, oxygen O₂, such molecule is not capable for participation in oxidative biological processes, for its activation heterogenous, energically stimulated “triplet-singlet” conversion ¹TO₂→³TO₂→¹SO₂ must occur, i.e. this process can only be triggered at the expense of catalytic properties of crystals and particles (TiO₂, SiO₂) by the way of transferring additional energy of 130-180 KJ/mol, i.e. at the expense of endothermic reaction with enthalpy (Janbazi H, Schulz C, Wlokas I, Peukert S. Thermochemistry of Oxygen-Containing Organosilane Radicals and Uncertainty Estimations of Organosilane Group-Additivity Values. J Phys Chem A. 2021 Oct. 7; 125 (39): 8699-8711. doi: 10.1021/acs.jpca.1c06941. Epub 2021 Sep. 24. PMID: 34559967).

Conversion conditions ¹TO₂→³TO₂→¹SO₂ obtaining of active, singlet oxygen in gaseous medium is easier to execute, for example, by the way of direct absorption of photons. In fluid medium such conversion is impossible. Another, special catalyst is necessary-thermosensor) manufactured in a special way in the form of crystals or SiO₂ particles.

Catalysis is thermally dependent: rising temperature also initiates a process of ¹TO₂→³TO₂→¹SO₂ conversion.

A mediator in liquid, aqueous medium in the organism, may be dense, not dissolved substance with maximum large developed surface, on which there are sufficient number of active energy centers such as crystals or particles. Sensor must be in heterophase condition (polymer, suspension) and for “triplet-singlet” conversion ¹TO₂→³TO₂→¹SO₂ it must have additional energy from without (E), no less than 130-180 KJ/mol.

Catalyst, photosensor must have electronically-excited form of oxygen molecule.

In the solution it is only possible by a contact catalysis reaction containing crystals and particles of TiO₂ or SiO₂ of a special QD class.

At the surface of heterocrystals and particles the energy centers (zones of local overheating) must be present, where oxygen molecule is in a third-stage triplet state (³TO₂);

The catalytic properties of thermosensor TiO₂ (ADAM-QD/T) or SiO₂ (ADAM-QD/S) provide the obtaining of active, singlet oxygen in the proposed invention is achieved for the first time from energy zones of the human body itself (36.6° C.). At contact of zones the energy of an organism becomes sufficient for transition from ³⁺TO₂ to ¹SO₂. Catalytic properties of crystals and particles of special class QD are unique in the field of creation of different medical product forms for influencing a wide range of pathogens and also in particular cases of diseases (pathologies) for reaching complex and local therapeutic effect. In options provide a simultaneous opportunity to carry out photon or hyperthermia or including electromagnetic wave and resonant emitter in order to achieve a synergistic therapeutic effect.

Table 7 shows the comparative data for: A. Photosensitizing products available on the market; B. photosensitizing substances of QD class TiO₂ crystals and SiO₂ particles; C. Thermosensor, catalyst for the synthesis of ROS (in the body) of QD class TiO₂ crystals and SiO₂ particles. * Fluorescence—where it demonstrates in dynamics the penetration and withdrawal from the cell of rhodamine-labeled luminescent stable dispersions of TiO₂ crystals and SiO₂ particles of QD class, using the example as indicated in FIG. 6.

As can be seen from Table 7, crystals and particles of TiO₂ and SiO₂ in the case of a photocatalyst and a thermal catalyst (B, C) have fundamental advantages over hypersensitive substances existing on the market pigments (A) (photocatalysts), in particular:
1. Lack of general toxicity;
2. Large surface of contact with the medium;
3. Wide possibilities of sorption bonds;
4. Colorlessness, transparency, intrinsic optical density;
5. Insolubility (creation of a heterogeneous suspension structure);
6. High thermal stability-protection from destruction under laser and other exposures;
7. Wide range of spectral photosensitivity;
8. Maximum catalytic, thermal activity also in the zone of low optical density of tissues;
9. The presence of active energy centers (zones of local overheating) of photo-thermo-catalysts, where on the surface of TiO₂ or SiO₂ oxygen (O₂) is in the electronically excited third-stage triplet state ³⁺TO₂;
10. Targeted therapeutic delivery in areas where pathogens occur in the human body.
11. A new class thermal sensitizer, Quantum Dots (QD), is activated by body temperature itself, producing the synthesis of reactive oxygen species.
12. Catalyst with the ability to simulate therapeutic effects as photosensor and/or thermosensor, providing the synthesis of ROS, using the exact physiological temperature of the body itself;
13. TiO₂ crystals and SiO₂ particles create an energy depot (days, weeks) in the local zone, which is destructive for the pathogen, by providing a continuous, permanent therapeutic process of AFO synthesis in the body.

Therapeutic provision of O₂ for the needs of the organism in acute situations is possible by any mobilization route available in medical practice.

But in chronic and dynamically developing pathogenic processes in the human body, an additional source of catalysis is required for introduction into cells deficient in natural means of activation of dissolved oxygen (PO₂). The process of AFO synthesis cardinal and fundamental for human life becomes critical in the body, especially in the presence of chronic diseases due to aging of the organism, as a consequence of the functioning of the systems that support the basis of ‘underoxidation syndromes’ (atherosclerosis, diabetes, oncology, neurodegenerative, orthopaedic diseases and other). As a consequence of a defect in the functioning of AFO synthesis in the body, providing ‘under-oxidation syndromes’-donor for AFO synthesis is needed. (see Examples 5,6).

For “complete oxidation” for a controlled therapeutic period, solid and insoluble crystals or particles are needed (catalyst), which ensure a continuous, permanent process of AFO thermosynthesis in the body.

An ideal catalyst, an exemplary semiconductor, which contains on its surface many energy centers in the form of outputs of semiconductor exciton channels, these outputs correspond to the location of oxygen molecules already in an electronically excited (EE) quasi-stable state, since they are included in the structure of the crystal lattice for these purposes-TiO₂, SiO₂ suspensions of a special Quantum Dots (QD) where in the structures of heterocrystals of oxides oxygen molecules occupy from 60-80% and are in an electronically excited third-stage triplet state (³⁺TO₂).

There are no test determinations of thermal thermoreceptors under laboratory experimental conditions, such as determinations of receptors in blood that moves, not in a blood vessel, but outside the body. Therefore, it is possible to demonstrate the effect of AFO synthesis and therapeutic effect at the stage of evidence-based preclinical studies, which is demonstrated in Examples 5,6,7,8, which confirm the complete oxidative process in the body.

By introducing a stable suspension of TiO₂ or SiO₂ into the body, we get a tool and the process of heat catalysis in the body in the liquid, aqueous medium of the tissues, by body catalyst, can be an undissolved, dense substance with a maximally large developed surface, with a sufficient number of active energy centres, they are the detonator of the transfer of unpaired electrons of the oxygen molecule from parallel to antiparallel orbits. Due to the participation of contact catalysis, the molecule accumulates a significant energy charge, which is then spent on oxidation with the formation of chemical compounds-radicals (R) for the treatment of metabolic disorders, respectively, the catalyst acts as a thermosensor. It means that adsorbed amounts of various excited states of oxygen are activated at physiological body temperature, synthesizing singlet form of oxygen ³TO₂→¹SO₂.

The development of irreversible pathologies in the human body requires significant amounts of oxygen (the enzyme NADP-H is an immune response of phagocytes), as well as stores of available oxygen for ROS synthesis. To mobilise the required dissolved O₂ and activate ROS synthesis, an oxygen donor must be applied. Oxygen adsorbed on the surface of TiO₂ crystals or SiO₂ particles is a suitable therapeutic source. NADPH-oxidase (NOX) is one of the main sources of cellular reactive oxygen species (ROS) and is formed by catalysis.

The flow of electrons from the excited energy reaction centres of TiO₂ crystals or SiO₂ particles is targeted to the enzyme complex NADPH oxidase which catalyzes the production of a superoxide free radical by transferring one electron to oxygen from NADPH.

(NADPH+2O₂↔NADP⁺+2O₂+H⁺)

Oxygen-derived free radicals, collectively termed reactive oxygen species (ROS), play important roles in immunity, cell growth, and cell signaling. In excess, however, ROS are dangerous to cells, and the overproduction of these molecules leads to a myriad of devastating diseases. (Panday A, Sahoo M K, Osorio D, Batra S (January 2015). “NADPH oxidases: an overview from structure to innate immunity-associated pathologies”. Cellular & Molecular Immunology. 12 (1): 5-23; Aliev G, Priyadarshini M, Reddy V P, Grieg N H, Kaminsky Y, Cacabelos R et al. Oxidative stress mediated mitochondrial and vascular lesions as markers in the pathogenesis of Alzheimer disease. Curr Med Chem 2014; 21:2208-2217; Sharma, Pallavi; Jha, Ambuj Bhushan; Dubey, Rama Shanker; Pessarakli, Mohammad (2012-04-24). “Reactive Oxygen Species, Oxidative Damage, and Antioxidative Defense Mechanism in Plants under Stressful Conditions”. Journal of Botany. 2012:1-26.). During this process, O₂ is transported from the extracellular space into the cell and H⁺ is exported.

In addition, in the electron transport chain, during the reaction of oxygen with reduced NADH compounds, the energy of redox reactions stimulates the synthesis of adenosine triphosphate (ATP), which is the main energy source for living cells.

The targeting and evidence-based therapeutic safety of the QD class of TiO₂ crystals or SiO₂ particles is due to the localisation of the inflammatory process induced, provoked by macrophages in response to pathogens of various nature, both viral and non-infectious aetiology. The generated ROS are toxic to cellular pathogens, the accumulated superoxide leads to oxidative processes that destroy pathogenic agents and molecules.

TiO₂ crystals or SiO₂ particles of the special QD class, due to the presence of many excited reactogenic centres, stimulate the production of ROS by enzyme complexes, thereby triggering an immune response. The properties of TiO₂ crystals or SiO₂ particles as a component of a pharmaceutical preparation with an effective function of synthesis of long-term activation of dissolved inert oxygen show that it is an exemplary semiconductor and acts as a donor of energy exchange reactions. At the same time, healthy cells do not undergo irreversible damage because, unlike tumour cells, virus-infected cells, healthy cells-possess more efficient antioxidant enzyme systems.

The thermosensor is in heterophase [³⁺TO₂] to achieve the conversion to the active oxygen form ¹⁻³SO₂ requires energy (E), to stimulate the triplet-singlet transition.

The oxidative potential is important to influence the pathological focus area.

The implementation of the present invention provides the possibility of activating AFO synthesis by thermal catalysis at a physiological body temperature.

So, the catalyst should contain electronically excited but necessarily triplet (³⁺TO₂) third state form of oxygen molecule, which corresponds to energy more than 92 KJ/mol, and then for the formation of singlet ¹SO₂ form of oxygen is required in the case of TiO₂ crystals and SiO₂ particles of the QD class of only up to 16 KJ/mol.

Action of suspension on the base of said TiO₂ crystals and SiO₂ particles is aimed to stimulation of natural processes at the cell level. AFO synthesized by the QD class crystals or particles initiates the process of influence upon malfunctioning cells, which allows the development of drug formulations, of all known pharmaceutical applications. (publication No. EP4272745A1).

Stable suspension of TiO₂ crystals or SiO₂ particles, due to synthesis of regulated active oxygen AFO, in the first place, is selectively directed into the malfunctioning cells of an organism, targeting the areas of inflammation, with infectious and non-infectious nature, in particular, interacting with membrane-associated ferment NADP-H, the immune cells of phagocytes and macrophages, causing in them numerous cytomorphologic changes (vacuoles in cytoplasm, fragmentation of membrane, abnormality of mitosis), thereby launching apoptosis or necrosis type of death of pathogenic cells (because the pathogenic cells have no effective antioxidant ferments).

Many works showed, that all the cells, forming the blood vessel system, enough intensively generate AFO as a response both to physical irritants (for example, to pulsating blood flow), and to hormonal factors, in particular, angiotensin. On this model the consequences of intensification of AFO production induced by regulatory factors were in detail studied. Hishikawa K., Oemar B. S., Yang Z., Luscher T. F. (1997). “Pulsatile Stretch Stimulates Superoxide Production and Activates Nuclear Factor-kappa B in Human Coronary Smooth Muscle.” Circulation Research, 81, 797-803. Krieger-Brauer H. I., Medda P. K., Sattel B., Kather H. (2000). «Inhibitory effect of isoproterenol on NADPH-dependent O₂ generation in human adipocyte plasma membranes is mediated by betagamma-subunits derived from G(s).» J Biol Chem, 275 (4), 2486-90.

Target action of penetration in malfunctioning cells of organism, reaching inflammation areas, of infectious and non-infectious nature is conditioned by multiple increase of oxygen necessity in inflammation focuses, in this case the targeted delivery of TiO₂ crystals or SiO₂ particles stable dispersions of a new QD class is provably demonstrated.

Example of targeted delivery of stable dispersions.

FIG. 6 shows an example of studying penetration into the cell of luminescent stable dispersions of heterocrystals of titanium dioxide crystals and silicon dioxide particles marked with rhodamine on the fibroblast cells of mice. Absorption of fluorescent QD by tumor cells of mice fibroblasts (NIH-3T3). From above, from the left to right, downwards in the course of time: confocal laser fluorescent microscopic images show, that a tumor absorbs QD, and then are eliminated from the cells.

The executed research with confocal laser microscope shows that luminescent crystals of the QD series marked with rhodamine penetrate inside the cell. Accumulation of the crystals initially occurs in cytoplasm. Cell nuclei accept the crystals in a less degree. In the top left section of the combined picture a normal transmission microscopic image of the treated cell with visible (deep black colour) crystals inside cytoplasm is visible. The images from the microscope were sequentially recorded, the treated cells were cut into layers with one micron thickness. It is important to note, that cutting of apical basal parts of the cell showed, that the number of luminescent crystals drops until total removal, also confirmed in Table 6.

### EXAMPLES

Examples of obtaining stable suspension TiO₂

### Example 1

The original nano-titanium dioxide powder in the form of aggregates 0.5 micrometers in size (modification of anatase and rutile taken in a ratio of 10:90) is a white powder, which, when studied in stability in accordance with the requirements of the International Council on Harmonization (ICH), showed that over time, the nanoparticles agglomerate, that is, the highly dispersed state is lost and stability decreases. Product has a characteristic odor of butanol and isobutanol; moreover, due to the residual solvent content, the starting material does not meet pharmacopoeial requirements. Preparation of solution 500 ml 0.1 N initial solution of HCl is prepared of aqueous solution HCl.

### Dispersion.

100.0 g of TiO₂ with water adsorbed (recalculated for dry mass) is weighed. Dispersion of TiO₂ is executed in 300 ml 0.1 N solution of HCl.

### Irradiation is Ultrasonic Bath

For preparation suspension is irradiated in ultrasonic bath working at frequency 20-90 kHz (bath temperature: 60° C., irradiation time: from 10-90 minutes).

During the process, the aqueous suspension is periodically sent to a hydrodynamic cavitation homogenizer.

Evaporation of dispersion medium.

Dispersion medium is steamed in vacuum-rotary evaporator.

During the evaporation of the dispersion medium, the final release of volatile hydrochloric acid vapor occurs, as well as the removal of residual amounts of volatile undesirable impurities (for example, butanol, isobutanol) to a level below the detection limit by gas chromatography (European Pharmacopoeia method).

### Filtration

The dispersion is filtered through a filter fabric made of polyethylene terephthalate threads with a mesh size of 5 micrometers (Sefar Medifab). The filter is examined during process control: through a microscopic assessment, the integrity of the filter is checked by observing possible mechanical damage.

### Evaporation of Dispersion Medium

Dispersion medium is evaporated in vacuum-rotary evaporator under pressure lower than 100 kPa at temperature no more than 70° C. In the process of evaporation of low pH dispersion medium, volatile vapors of hydrochloric acid are discharged meanwhile cleaning the surface of TiO₂ nanoparticles, thereby activating as the chemical properties at the surface undergo significant changes. After evaporation of dispersion medium, dry activated powder TiO₂ compliant with GMP and FDI requirements is obtained.

When releasing variations of aqueous solutions according to GMP requirements the powder of activated TiO₂ crystals of the new QD class is directed into the hydrodynamical cavitational homogenizer, in which water is added for obtaining from 0.001% to 10% stable suspension TiO₂ and expose to additional treatment during 10-90 minutes.

Stability in time for suspension obtained in such a way is from 24 months and more than 15 years without changes of optical properties. (FIG. 2)

### Example 2

Initial powder of nanosized titanium dioxide (modification of anatase and rutile taken in ratio 10:90) is white powder in the form of aggregates with size 0.5 micrometer with a characteristic smell of butanol and isobutanol, when studying stability in accordance with the requirements of the International Council for Harmonization (ICH), it was found that over time the nanoparticles agglomerate, that is, they lose their highly dispersed state and stability decreases. In addition, the odor of the solid material is undesirable, i.e. the product has a characteristic odor of butanol and isobutanol; moreover, due to the residual solvent content, the starting material does not meet pharmacopoeial requirements.

Preparation of solution—500 ml 0.1 N initial solution of HCl is prepared of aqueous solution HCl.

### Dispersion.

100.0 g of TiO₂ (recalculated for dry mass) is weighed. Dispersion of TiO₂ is executed in 300 ml 0.1 N solution of HCl.

### Irradiation is Ultrasonic Bath

For preparation suspension is irradiated in ultrasonic bath working at frequency 20-90 kHz (bath temperature: 60° C., irradiation time: 1.2 hours).

### Filtration.

The dispersion is filtered through a filter fabric made of polyethylene terephthalate threads with a mesh size of 5 micrometers (Sefar Medifab). The filter is examined during the inspection process: through microscopic evaluation, the integrity of the filter is checked by observing possible mechanical damage.

### Evaporation of Dispersion Medium.

The dispersed medium is evaporated in a vacuum rotary evaporator under a pressure below 100 kPa. at a temperature of not more than 70° C. During the evaporation of the pH of the dispersion medium, volatile vapors of hydrochloric acid are released meanwhile the surface of TiO₂ nanoparticles is thereby activated as the chemical properties at the interface undergo significant changes. After evaporation of the dispersion medium, dry activated TiO₂ powder is obtained. In addition, evaporation helps remove residual amounts of volatile unwanted impurities (e.g. butanol, isobutanol) to levels below the detection limit using gas chromatography (European Pharmacopoeia method) In the manufacturing of various options for aqueous dispersions in accordance with GMP requirements, activated crystal powder TiO₂ of the new QD class is loaded into the hydrodynamical cavitational homogenizer, in which water is added for obtaining from 0.01% to 10% stable suspension TiO₂ and expose to additional treatment during from 10 to 90 minutes (1 minutes from 10 liters of treated suspension, where content depends on the specified indices, i.e. 0.001-10%).

Stability in time for suspension obtained in such a way is at least 24 months. During not less than 15 years changes in particle size distribution were not indicated.

Example of obtaining stable suspension SiO₂

### Example 3

Silicon dioxide nanopowder was investigated in a stability study in accordance with the International Council for Harmonisation (ICH) requirements. During the study it was found that the nanoparticles agglomerate, i.e. lose their highly dispersed state and reduce stability, resulting in non-compliance with the particle size stability requirements. 255 g of SiO₂ powder is dispersed in 10 l of water adding 0.0001 N of HCl solution. Then dissolved nano-powder is placed into a reaction vessel with volume of 5000 ml, where dispersion is executed under pressure 900 kPa-1200 kPa with speed 10 l/min.

### Irradiation is Ultrasonic Bath.

For preparation suspension is irradiated in ultrasonic bath working at frequency 20-90 kHz (bath temperature: 60° C., irradiation time: 1.2 hours).

Suspension of SiO₂ particles is obtained with particles size in 95.0 number %<250 nm. Content of silicon dioxide is 50 mg/ml.

Reactionary mixture is filtered through a mesh filter 10 micrometer (Sefar Medifab).

After filtration all the volume of suspension is pumped into a glass container and crimped thoroughly.

Obtained suspension is placed into an autoclave at temperature 121° C., exposure time: 30 min.

### Evaporation of Dispersion Medium.

Dispersion medium is evaporated in vacuum-rotary evaporator under pressure lower than 10 kPa at temperature not more than 70° C.

In the process of evaporation of low pH dispersion medium volatile vapors of hydrochloric acid and other impurities are discharged. Meanwhile, surface of SiO₂ particles is cleaned and activated, as chemical properties at the surface undergo significant changes. After evaporation of dispersion medium, dry activated powder SiO₂ compliant with GMP and FDI requirements is obtained.

When releasing variations of aqueous solutions according to GMP requirements circulation of mixed aqueous mixture of silicon dioxide of the new QD class through a suspending device in the hydrodynamical cavitational homogenizer with input pressure 900 kPa-1200 kPa during up to 90 minutes at room temperature.

Appearance—white or gray-white suspension, shaken up translucent, but orange tinted, easily resuspended.

Size of particles EP 2.9.31 min 90.0 number %<250 nm. Content of silicon dioxide: 50 mg/ml, pH of the product: 5.0-7.5, dynamic viscosity: 0.94-1.14 mPas.

Stability in time for suspension obtained in such a way is at least 24 months. During not less than 15 years changes in particle size distribution were not indicated (FIG. 3).

### Example 4

Calculation of the Presence of Oxygen on the TiO₂ Crystals and SiO₂ Particles Surface.

The particle size distribution (of TiO₂ or SiO₂) was determined by dynamic light scattering (photon correlation spectroscopy) by using a Malvern Zetasizer Nano ZS instrument applied with a laser source emitting laser beam of wavelength □□=633 nm at 25° C. in aqueous dispersions diluted to 0.6 mg/mL concentration. The instrument provides intensity, volume and number weighted distribution profiles.

In order to obtain the surface-weighted size distribution in case of spherical crystals or particles for evaluation of sorption features related to different energetic states of dissolved oxygen, from the volume-weighted distribution, the following formula was used:

$${A}_{iS} ( \% ) = \frac{{V}_{iS} ( \% )}{{D}_{i}} \times \frac{100}{\sum \frac{{V}_{iS} ( \% )}{{D}_{i}}} ,$$

wherein
A_{iS}(%) is the area corresponding to fraction i of spherical crystals or particles expressed in % of total area;
V_{iS}(%) is the volume corresponding to fraction i of spherical crystals or particles expressed in % of total volume;
Dᵢ is the diameter of fraction i crystals or particulates.

However, for non-spherical crystals or particles of the new Quantum Dots (QD) class according to present invention different calculation method for the surface-weighted size distribution is required, i.e. using the following formula to have insight into the sorption properties with relevance to different O₂ species according to present invention:

$${A}_{iQD} ( \% ) = \frac{{V}_{iQD} ( \% ) \times {F}_{i}}{{D}_{i}} \times \frac{100}{\sum \frac{{V}_{iQD} ( \% )}{{D}_{i}}} ,$$

wherein
A_{iQD}(%) is the area corresponding to fraction i of non-spherical crystals or particles expressed in % of total area;
V_{iQD}(%) is the volume corresponding to fraction i of non-spherical crystals or particles expressed in % of total volume;
Fᵢ is the individual shape factor (non-sphericity parameter) corresponding to fraction i
Dᵢ is the diameter of fraction i crystals or particles.

Exemplary calculations for a known nanosized TiO₂ disclosed in PCT patent application WO2008068062 in comparison with the TiO₂ crystals of the new quantum dots QD class according to present invention are shown in Table 8, which shows conversion of surface-weighted size distribution from volume-weighted distribution

Spherical crystals having AiS (%) distribution profile as shown in Table 8 according to WO2008068062 represents 5 micromol/m2 adsorptive capacity and 90 m2/g specific surface area. 1 g of such nanosized TiO₂ hence possesses 450 micromol/g adsorptive capacity which is equal to 0.0144 g 02/g TiO₂ (1.44% by weight). Non-spherical crystals of the special QD class according to present invention show significantly higher AiQD (%) at smaller particulate size population (i.e. 20 nm) which allows adsorbing more reactive form of oxygen in the amount of about 60-80% is in electronically excited triplet state.

The TiO₂ crystals of the QD class according to the present invention are a group of irregular, prismatic shape with rectangular faces, sometimes with pointed ends, and in the case of QD class SiO₂ they have a crystalline structure and form interfaces and, as in the case of QD class TiO₂, have the same narrow contact zones with each other, i.e. the (secondary) nanodispersed particles are formed by agglomeration already after the preparation of the suspension, the stability of which is indicated in Tables 2 and 4. The increased surface area due to the nonsphericality allows to increase the adsorption capacity for different excited states of oxygen.

Taking into account the new structure of crystals and particles of the QD class, containing on their surface many energy centres in the form of exciton semiconductor channel outlets, these outlets correspond to the location of oxygen molecules already in the electronically excited (EE) state, since they are included in the crystal lattice structure-which appear as a special class of quantum dots (QD), where in the structures of oxide heterocrystals oxygen molecules occupy from 60-80% and are in the electronically excited triplet state (³⁺TO₂).

The present invention relates to stable suspensions of titanium dioxide or silicon dioxide heterocrystals, methods of obtaining said quasi-stable suspensions and use provide more effective delivery of biologically active agents into the bloodstream of a subject, accordingly distributed throughout the body as conclusively demonstrated in Table 6, sufficient for causing a specified biological reaction, that is, a therapeutically effective amount of a therapeutic agent is meant. What is meant is a pharmaceutically active substance sufficient to produce an acceptable biological response when administered. Correspondingly, TiO₂ or SiO₂ suspensions may be delivered with a help of usual micro-fluidized spray, hydrogel, topical products, aerosol or liquid. Delivery may be executed by parenteral, intrathecal, intravenous, intravascular, droplet, transmembrane and dermal, intracellular administration including locally into cell structures through mucous membranes, and also due to their structures and range limits provide new models-forms of administration or every other generally recognized method of medical agent's delivery.

A peculiarity of TiO₂ crystals and SiO₂ particles is that on their surface an acceptor, i.e. O₂, is initially in excited triplet triplet third stage (³⁺TO₂) condition, with regulated possibility of catalysis in AFO, even in the cases where physiological body temperature is sufficient.

Heterogeneous crystals of TiO₂ or SiO₂ particles are characterized by presence on the surface of oxygen, O₂, which occupies from 60 to 80% of structure have biological activity, solution by thermal synthesis of AFO, achieved for the first time due to the human body temperature 36.6° C. itself, where it creates a therapeutic energy storage (days, weeks), which ensures a continuous, permanent process of AFO synthesis in the body.

Medical forms on the base of titanium dioxide crystals or silicon dioxide particles, as catalyst of synthesis of active oxygen (AFO), firstly, selectively penetrate into the pathogenic cells of organism, targeting the areas of inflammation, with infectious and non-infectious nature (see Examples 7, 8), in particular, the cells using a ferment NADP-H for AFO formation, phagocytes and macrophages, causing in them numerous cytomorphologic changes (vacuoles in cytoplasm, fragmentation of membrane, abnormality of mitosis), that launches apoptosis or necrosis type of death of pathogenic cells (because the pathogenic cells do not have effective antioxidant ferments). Active oxygen, AFO become firstly claimed in the pathologies in focus (Examples 5,6,7,8,9), where oxygen necessity increases many times.

NADP-H oxydases membrane-bound enzymatic complex, inversed into extracellular space of plasmatic membrane, and also in membranes of phagosomes, used by neutrophilic leukocytes (immune cells, white blood corpuscles) for absorption of microorganisms is executed with the immune response. (See Example 8).

NADP-H oxidase (NOX) is one of the main sources of cellular active forms of oxygen (AFO) and is formed by the way of catalysis.

Accordingly, when oxygen (O₂), on crystal lattices is initially in the excited state ³⁺TO₂ in cases of form, topical use in the form of patches, impregnations for tampons, pads, diapers, tapes, bandages, etc., which have antimicrobial and antiseptic effects, anti-inflammatory, neuroprotective and immuno-normalizing effects, including adsorption effects, as well as mucosal forms of application, thereby creating a new modality of effective therapy.

The mechanism of action where TiO₂ crystals or/and SiO₂ particles are present in the composition of a medical product is that when electronically excited O₂ interacts on the surface of crystals or particles with the membrane cellular enzyme complex NADPH, AFO is formed, providing energy exchange in the body. The energy returned to the organism, passing through the nerve impulses, is converted into an action potential. The action potential at the presynaptic synapse depolarises the presynaptic membrane, causing a rapid current of Ca²⁺ ions. The temporary increase in the concentration of Ca²⁺ ions stimulates the fusion of the synaptic vesicle membrane with the plasma membrane and causes the release of the mediator, for example—serotonin, into the synaptic cleft. The interaction of mediator-serotonin in the synaptic cleft with its chemoreceptor in the postsynaptic membrane changes the membrane permeability for (Na⁺) ions, which provides normalisation of serotonin receptor function, restoring the serotonin balance of the organism.

Efficiency of using stable suspensions of TiO₂ heterocrystals and SiO₂ particles for adjuvant therapy of cancer diseases is also experimentally proven in combination with cytostatic agents (Doxorubicin, Lenalidomide (Revlimid), Nivolumab, Ibrutinib).

A positive result was demonstrated on the model of metastasis into spleen and liver, primary tumor significantly reduces after intra-abdominal administration, after administration together with cytostatic agent. In addition, a tendency to decrease of metastatic nodes was observed.

The experiments have proven (see Examples 5-8) the use of stable suspension of heterocrystals of titanium dioxide or silicon dioxide particles, for formulation of medical agents, used intravenously, intramuscularly, orally, nasally, vaginally, rectally, locally (ENT) use or topically, at that therapeutically important amount of an active substance is for intravenous use from 0.006 mg/ml, intramuscular use from 0.01 mg/ml, orally from 0.01 mg/ml, nasally from 0.01 mg/ml, vaginally from 0.01 mg/ml, rectally from 0.01 mg/ml, local ENT use from 0.01 mg/ml, ocular use from 0.001 mg/ml, and also medical articles of topical use in the form of hydrocolloidal medical plasters from 0.0003 mg/g. (patent WO2023/214200).

Usually, therapeutically effective amount of a medical means is meant. The term “therapeutically effective amount” means that pharmaceutically efficient amount is considered in relation of, for example, pharmaceutical preparations. Pharmaceutically efficient amount is amount of a medical remedy or pharmaceutically active substance that is sufficient for achieving acceptable biological reaction in its use.

A medical remedy may be executed in the form of rectal-vaginal suppositories, gel, ointment, liquid.

THE FOLLOWING EXAMPLES OF EFFECTIVE IMPACT NOT ONLY ON THE DISEASE (PATHOLOGY) EXAMPLE, BUT ILLUSTRATE A WIDE RANGE OF POSSIBILITIES OF EFFECTIVE THERAPY OF VARIOUS NOSOLOGIES OF MEDICAL PRODUCT APPLICATION FOR ELIMINATION OF UNDEROXIDATION PROCESSES IN LIVING ORGANISM DUE TO AFO SYNTHESIS.

### Example 5

5.1. In Vitro Studies Related to Alzheimer's Disease with the Use of SiO₂ Particles of the New QD Class (Adam-QD/S)

### In Vitro Studies

### Study Number QD-2023-1

The purpose of this in vitro interaction study is to explore the mechanism of action of the SiO₂ particles to exert its therapeutic effect via a the peroxidase-mimetic catalytic effect of the particles using 3,3′,5,5′ Tetramethylbenzidine (TMB) reagent, which plays the role of the reducing agent (hydrogen donor) in the reduction of H₂O₂ to water.

Test samples: Representative SiO₂ particles of the QD class characterized by volume and surface weighted distribution per FIG. 4 were used for the studies. Various amounts of SiO₂ particles were weighed into a 25 ml volumetric flask to reach 0.024-0.06 mg/ml concentration, filled it with 20 ml of purified water, and then mixed it with 2.5 ml of a 2 mg/ml TMB ethanolic solution. The pH of the mixture was adjusted to 3.0, 3.5, 4.0, respectively with 1 N hydrochloric acid solution, then filled the flask to the mark with purified water.

Blank sample was prepared analogously without SiO₂.

The Test and Blank samples were transferred separately into a 50 ml beaker, placed them in a 39-40° C. water bath and stirred them in air at a speed of 500 RPM. The spectrophotometric analysis of the samples was performed with an Agilent 8453 spectrophotometer, applying a quartz cuvette with an edge length of 1 cm, at a wavelength of 650 nm. The absorbances of the Test samples were measured without dilution, directly against the Blank sample.

The results showed that in the presence of nanoparticulate SiO₂ the rate of oxidation of TMB (3,3′,5,5′ tetramethylbenzidine) was significantly enhanced in concentration dependent manner in the range of 0.024-0.06 mg/mL (30-60-fold). At pH 4, the rate of oxidation approximately tripled compared to what was observed at pH 3.

5.2. In Vitro Studies Related to Alzheimer's Disease with the Use of TiO₂ Crystals of the New QD Class (Adam-QD/T)

### Study Number QD-2023-1

The purpose of this in vitro interaction study is to explore the mechanism of action of the TiO₂ crystals to exert its therapeutic effect via the peroxidase-mimetic catalytic effect of the crystals using 3,3′,5,5′ Tetramethylbenzidine (TMB) reagent, which plays the role of the reducing agent (hydrogen donor) in the reduction of H₂O₂ to water.

Test samples: Representative TiO₂ crystals characterized by volume and surface weighted distribution per FIG. 4 was used for the studies. Various amounts of TiO₂ crystals were weighed into a 25 ml volumetric flask to reach 0.024-0.06 mg/ml concentration, filled it with 20 ml of purified water, and then mixed it with 2.5 ml of a 2 mg/ml TMB ethanolic solution. The pH of the mixture was adjusted to 3.0, 3.5, 4.0, respectively with 1 N hydrochloric acid solution, then filled the flask to the mark with purified water.

Blank sample was prepared analogously without TiO₂.

The Test and Blank samples were transferred separately into a 50 ml beaker, placed them in a 39-40° C. water bath and stirred them in air at a speed of 500 RPM. The spectrophotometric analysis of the samples was performed with an Agilent 8453 spectrophotometer, applying a quartz cuvette with an edge length of 1 cm, at a wavelength of 650 nm. The absorbance of the Test samples was measured without dilution, directly against the Blank sample.

The results showed that in the presence of TiO₂ crystals the rate of oxidation of TMB (3,3′,5,5′ Tetramethylbenzidine) was significantly enhanced in concentration dependent manner in the range of 0.024-0.06 mg/mL (25-50-fold). At pH 4, the rate of oxidation approximately doubled compared to what was observed at pH 3.

### 5.3. In Vitro Studies (Yeast Cell Study) in the Anti-Aging and Oxidative Stress by Using TiO₂ Crystals of the New QD Class (ADAM-QD/T)

### Study Number QD-2024-1

The purpose of this in vitro interaction study is to explore the mechanism of action of the TiO₂ crystals, how it exerts its therapeutic effect. It is postulated that these crystals prevent oxidative stress by deactivating oxidative agents such as H₂O₂. To achieve the aim, a biorelevant study on pH- and concentration dependence of redox catalytic activity i.e. peroxidase-mimetic effect of the corresponding TiO₂ crystals was modelled using a model dye, MTT Formazan (1-(4,5-dimethylthiazol-2-yl)-3,5-diphenylformazan) which reagent plays the role of the reducing agent (hydrogen donor) in the reduction of H₂O₂ to water. Peroxidases are important enzymes in antiaging and combat oxidative stress.

MTT is the most commonly used colorimetric assay for assessing cell metabolic activity usually used to measure cytotoxicity (loss of viable cells) or cytostatic activity of potential medicinal agents. NADPH-dependent cellular oxidoreductase enzymes reflect the number of viable cells present. These enzymes are capable of reducing the MTT dye which has a purple colour which enables the recordation of the kinetics photometrically.

Representative TiO₂ crystals characterized by volume and surface weighted distribution per FIG. 4 was used for the studies.

Liquid samples were prepared containing constant amount of MTT formazan, set to target pH, containing various concentrations of nanosized TiO₂ (0.006-0.12 mg/mL), hydrated live yeast cells (0.023 mg/mL on dry cell basis) and fixed amount of H₂O₂ (in 1.5 M concentration). The samples were stirred in a dark cabinet thermostated close to body temperature. The samples were repeatedly analyzed with 30 min frequency in a total of four hours interval with Agilent 8453 spectrophotometer.

In 25 ml screw cap scintillation vials, 8.00 ml 96 V % ethanol, 4 ml TiO₂ crystals suspension (set to different concentration), 3.00 ml of 0.25 mg/ml ethanolic MTT formazan solution, finally purified water, finally hydrated live yeast cells (0.023 mg/mL on dry cell basis) were added in this order. After optional pH setting with 1% Na₂HPO₄, 4 ml 30% H₂O₂ reagent was added. The kinetic curves obtained during the study (i.e. the decreasing concentration of MTT formazan) of the oxidative process were recorded spectrophotometrically.

It was found that under the used experimental conditions, TiO₂ crystals can facilitate the reduction of H₂O₂ by catalyzation of a biologically relevant electron transfer reaction. Deactivation of H₂O₂ is well known to have therapeutic relevance and also may be desirable in various life science applications. The model reaction represents peroxidase-mimetic effect, wherein MTT formazan is the hydrogen donor hence it reveals the mechanism how TiO₂ crystals combat oxidative stress.

TiO₂ crystal was found effective in the concentration range 0.006-0.12 mg/ml in concentration dependent manner reaching maximum activity at 0.06 mg/ml. The effect of TiO₂ has been well demonstrated at pH 6 and 8 (the latter value is mitochondrial pH).

5.4. In Vivo Studies Related to Alzheimer's Disease with the use of SiO₂ Particles of the New QD Class (ADAM-QD/S)

### Study Number: 9160

Efficacy of SiO₂ particles after intramuscular dosing of 5×FAD mice.

The aim of this study was to test the efficacy of SiO₂ particles after intramuscular dosing of 5×FAD mice. A total of 12 female transgenic 5×FAD mice at an age of 7 months, as well as 6 age- and sex-matched wildtype littermates were used for the study. After finishing behavioral testing, when the animals reached an age of 10 months, all mice were euthanized. Cerebrospinal fluid and terminal blood were collected. Brains were dissected after transcardial perfusion with saline and hemisected at midline. The left hemibrain was further dissected in hippocampus, cortex and rest, all parts were weighed and snap frozen on dry ice for biochemical analysis. The right hemibrain was post-fixed by immersion in 4% PFA, and cryo-protected in sucrose/PBS before embedding and freezing in cryomolds for histological evaluations. NF-L levels were quantified in plasma and CSF samples from all animals (18 samples) using the NF-Light® ELISA. Measurements were performed in duplicates. Hemibrains from 6 animals per group (total of 30 hemi brains) dedicated for histological analysis were embedded in sagittal orientation in OCT medium and 10 μm cryosections were collected. Sections were collected from 12 levels of the brain and 5 sections per level, a total of 60 sections per animal. Amyloid-β-positive plaques, tau phosphorylation (Ser202, Thr205) and neuroinflammation (astrocytosis and astrogliosis) were evaluated using immunofluorescence labeling on a uniform systematic random set of five sections per mouse in a quadruple staining:

Representative SiO₂ particles characterized by volume and surface weighted distribution per FIG. 4 was used for the studies.
mouse anti amyloid-β monoclonal antibody [6E10]
Phospho-Tau (Ser202, Thr205) monoclonal antibody
GFAP (astrocytosis)
Iba1 or CD11b (activated microglia)

All sections were counterstained with the nuclear dye DAPI.

### Outcome:

General: All animals tolerated the treatment. Neither the treatment route nor treatment with the test item led to unusual occurrences. The mean body weight of all groups increased over time as expected, whereas non-transgenic animals showed a typically higher body weight compared to transgenic animals without reaching significance at all times. Behavior: Statistical significances could be detected in learning curves throughout all evaluated parameters in these two groups.

Treatment with SiO₂ particles significantly increased the mean size of detected objects in the cortex; this indicates that SiO₂ particles reduces the formation of new (small) plaques as shown by βAmyloid signal detection. B.5×FAD vehicle group analysis showed 160±5 μm² cortex object sizes, whereas SiO₂ particles treated group analysis resulted in 180±5 μm² cortex object sizes.

5.5. In Vivo Studies Related to Alzheimer's Disease with the Use of TiO₂ Particles of the New QD Class (Adam-QD/T)

### Study Number 0430

The aim of the study is to examine the effect of titanium dioxide crystals on cytotoxicity and mitochondrial activity in neural cells upon H₂O₂ lesion in two different cell systems, i.e. cortical glutamatergic neurons, microglia and astrocytes.

Dysfunction of cortical glutamatergic neurons may be relevant to neurological diseases, such as Alzheimer's disease.

Microglial degeneration and death have been reported in research on schizophrenia and Alzheimer's disease.

Clinically significant pathologies involving astrocytes include astrogliosis and astrocytopathy associated with multiple sclerosis, anti-AQP4+ neuromyelitis optica, Rasmussen's encephalitis, Alexander disease, and amyotrophic lateral sclerosis. Studies have shown that astrocytes may be implied in neurodegenerative diseases, such as Alzheimer's disease, Parkinson's disease, Huntington's disease, Stuttering and amyotrophic lateral sclerosis.

The study involved determination the EC50 of H2O2 for four read-outs: mitochondrial activity, cytotoxicity, mitochondrial singlet oxygen imaging and ATP assay. Instrument used for quantitation: Incucyte® Live-cell imaging system.

Overall, neurons were found vulnerable to H₂O₂ of 100-1000 μM concentration. TiO₂ crystals were able to reduce cell death and to increase mitochondrial health for cortical glutamatergic neurons and microglia. For astrocytes, temporary increased mitochondrial activity could be observed after lesion addition lasting for 10 hours.

Representative TiO₂ crystals characterized by volume and surface weighted distribution per Example 4 was used for the studies.

For astrocytes, the total intensity (expressed as OCU×μm²/image) as shown by the TMRM test in the presence of 100 μM H₂O₂ and 6 μg/mL, 60 μg/mL, 600 μg/mL of TiO₂ crystals was 1.5-2 times higher than the lesion control.

The cortical glutamatergic neurons, lesion of 300 μM H₂O₂ was detrimental, however, in the presence of 6-600 μg/mL TiO₂ crystals the total intensity 1.0-1.5 million OCU x μm²/image remained in the TMRM test.

For microglia, lesion of 1000 μM H₂O₂ was caused the TMRM signal from 4 million to 500000 OCU×μm²/image within 20 hours incubation, however, in the presence of 6-600 μg/mL TiO₂ crystals the total intensity of 2-7 million OCU×μm²/image remained in the TMRM test.

The results clearly indicate the neuroprotective effect exerted by the applied TiO₂ QD class crystals against cellular damage caused by aggressive, H₂O₂ lesion. Oxidative stress was highly reduced owing to the presence of TiO₂ which offers notable potential in therapeutic or preventive applications for neurodegenerative conditions. Hindering the oxidative potential of H₂O₂ is the mechanism of action manifested in anti-Alzheimer action of TiO₂.

### Example 6

6.1. In Vitro Studies Related to Osteoarthritis with the Use of TiO₂ Crystals of the New QD Class (Adam-QD/T)

### Study Number: L-03

The objective of the study was to effects of TiO₂ crystals on differentiation of mouse osteoblasts and differentiation and activity of human osteoclasts in vitro.

Osteoblasts and osteoclasts are specialized cells that play crucial role in bone development and regeneration. Osteoblasts form new bones and add growth to existing bone tissue. Osteoclasts remove old and damaged bone tissue so it can be replaced with new, healthier cells created by osteoblasts. Both cells differentiate (develop) from special stem cells. The below study is aimed to model how the TiO₂ test item is able to cause alteration of metabolic pathways i.e. eliminating underoxidized metabolic byproducts from bone tissue causing osteoarthritic conditions. Abnormal joint tissue metabolism is associated with the formation of unwanted byproducts that are produced abundantly in osteoarthritic articular tissues, their levels are higher in osteoarthritic condition compared to normal and when the oxidative homeostasis of articular cartilage becomes unbalanced.

In the osteoblast differentiation assay, KS483 mouse osteoprogenitor cells were cultured for 8 days, after which the formed osteoblasts were quantitated by measuring the amount of intracellular alkaline phosphatase (ALP) activity. Bone morphogenetic protein 2 (BMP-2) was included in the study as a reference compound that stimulates osteoblast differentiation and bone formation to demonstrate that the culture system works as expected. Test compound and the reference compound BMP-2 were added at day 0 (when cells are seeded) and at day 4 concomitant with medium was refreshment. The osteoclast differentiation assay was performed using human bone marrow-derived CD34+ osteoclast precursor cells. The cells were cultured on bovine bone slices for 7 days in conditions favoring osteoclast differentiation and allowed to differentiate into bone-resorbing osteoclasts. Denosumab was included in the study as a reference compound to demonstrate that the test system can detect inhibition of osteoclast differentiation. The test compound and the reference compound denosumab were added at day 0 (when cells were seeded) and at day 3 concomitant with medium refreshment. Tartrate-resistant acid phosphatase 5b (TRACP 5b) activity was measured in the culture medium collected at day 7 as an index of the number of osteoclasts formed during the differentiation period. For the osteoclast activity assay, human bone marrow derived CD34+ osteoclast precursor cells were cultured on bovine bone slices for 10 days in conditions favoring osteoclast differentiation. Odanacatib was included in the study as a reference compound to demonstrate that the test system can detect inhibition of osteoclast activity. The test compound and the reference compound odanacatib were added into the culture medium at day 7, and the formed osteoclasts were allowed to resorb bone for 3 days. C-terminal crosslinked telopeptides of type I collagen (CTX-I) was measured in the culture medium at day 10 as an index of bone resorption. The tests were performed in 96-well plates containing a baseline group with vehicle and a control group including the reference compound (BMP-2/denosumab/odanacatib). Seven concentrations of the test compound were included.

TiO₂ products of the QD class decreased mouse osteoblast differentiation significantly at 50-1000 μg/ml concentration range (from 90 to 75%) while 0.05-5 μg/ml concentration also caused decrease (from 15 to 10%).

TiO₂ products of the QD class decreased human osteoclast differentiation significantly (TRACP 5b) at 50-1000 g/ml concentration range (from 99 to 59%) while 5 ng/ml-5 μg/ml also caused decrease (from 11 to 19%).

TiO₂ products of the QD class decreased human osteoclast resorption activity significantly at 1000 μg/ml (by 73%) and while the effect is moderate at 5 ng/ml to 500 μg/ml concentration (by 27 to 11%).

Instrument used: VICTOR2™ Multilabel Counter (PerkinElmer, Waltham, MA, USA).

The results show that the differentiation of both cells can be modulated by the presence of TiO₂ crystals of the QD class (ADAM-QD/T) which has promising potential in treatment of osteoarthritic conditions with involvement of eliminating/preventing the formation of underoxidized metabolic byproducts before their early stages of occurrence.

6.2. In Vitro Studies Related to Osteoarthritis with the Use of SiO₂ Particles of the New QD Class (Adam-QD/S)

### Study Number: L-02

Osteoblasts and osteoclasts are specialized cells that play crucial role in bone development and regeneration. Osteoblasts form new bones and add growth to existing bone tissue. Osteoclasts remove old and damaged bone tissue so it can be replaced with new, healthier cells created by osteoblasts. Both cells differentiate (develop) from special stem cells. The below study is aimed to model how the SiO₂ test item is able to cause alteration of metabolic pathways i.e. eliminating underoxidized metabolic byproducts from bone tissue causing osteoarthritic conditions. Abnormal joint tissue metabolism is associated with the formation of unwanted byproducts that are produced abundantly in osteoarthritic articular tissues, their levels are higher in osteoarthritic condition compared to normal and when the oxidative homeostasis of articular cartilage becomes unbalanced.

Instrument used: VICTOR2™ Multilabel Counter (PerkinElmer, Waltham, MA, USA).

SiO₂ particles decreased osteoblast differentiation significantly (ALP specific activity) at 5-1000 μg/ml concentration (by 80 to 52%), however the 5 ng/ml caused 15% increase compared to the baseline.

SiO₂ products of the QD class decreased osteoclast differentiation significantly (TRACP 5b) at 50 ng/ml and 0.5-1000 μg/ml concentration (by 100 to 19%) and the 5 ng/ml decreased by 14% compared to the baseline.

SiO₂ products of the QD class decreased osteoclast resorption activity significantly (CTX-I) at 50-1000 μg/ml concentration (by 96 to 55%), and it caused 28% decrease in 50 to 5 ng/mL compared to the baseline.

The results show that the differentiation of both cells can be modulated by the presence of SiO₂ particles of the QD class (ADAM-QD/S) which has promising potential in treatment of osteoarthritic conditions with involvement of eliminating/preventing the formation of underoxidized metabolic byproducts.

6.3. IN VIVO Studies Relating to Osteoarthritic Changes with the Use of SiO₂ Particles of the New QD Class (ADAM-QD/S)

The effects of SiO₂ particles of the QD class on osteoarthritic changes induced by unilateral surgical medial meniscal tear and medial collateral ligament transection in rats

The objective of this study is to investigate impact of the SiO₂ particles on osteoarthritic pain and histological changes in knee joints exhibiting unilateral medial meniscal tear and medial collateral ligament transection in young adult male rats.

Each group contained 12 male Lewis rats that were three months of age at the beginning of in-life phase of the study. Before the in-life phase, the rats were trained for pain measurement devices (hind paw weight distribution and paw withdrawal threshold.

Instrument of analysis: CatWalk XT computer-assisted method of locomotor analysis (Noldus Information Technology, Wageningen, The Netherlands).

Test item in the form of SiO₂ suspension of the QD class was administered invasively (into the tight muscle-musculus quadriceps femoris—of the right operated hind limb and also, non-invasively, via a form of hydrocolloidal medical plaster. Upon invasive application, the vehicle solution was administered into the same area of the non-operated left hind leg to avoid treatment-induced differences in pain measurement between the legs. Upon non-invasive application, a medical article of topical use in the form of hydrocolloidal medical plasters containing nanosized SiO₂ was applied on the right hind leg of a separate group of test animals for 19 days. Control animals had unloaded (blank) plasters adhered to their right hind leg. Both groups had unloaded (blank) plasters adhered to their left hind legs also to avoid false conclusion on their modified movement pattern.

The test compound dosing suspension was 0.64 mg/ml, and dosed intramuscularly in thigh muscle of the right leg as 0.045 mg/kg and dosing volume 0.070 ml/kg.

Static mechanical allodynia was determined using filament response data measured as paw withdrawal threshold. After the operations and invasive treatment period with SiO₂ suspension, the test compound significantly reduced statistic mechanical allodynia on study days 7, 13 and 19. SiO₂ product of the QD class (ADAM-QD/S) has demonstrated positive effects on reducing static mechanical allodynia and static knee joint discomfort/pain in the test system. Compared to the vehicle control, the pain tolerability threshold increased twofold after 7 days followed by treatment with SiO₂ product of the QD class (ADAM-QD/S) and threefold after 13 and 19 days post treatment.

Throughout the non-invasive treatment period, it was also found that the SiO₂ particles taking effect from the plaster matrix, also have shown significantly reduced statistic mechanical allodynia on the study days (7th, 13th and 19th day). Even in non-invasive application, the SiO₂ product has demonstrated positive effects on reducing static mechanical allodynia and static knee joint discomfort for the test animals. Compared to the unloaded plaster control, the pain tolerability threshold increased twofold after 7 days followed by non-invasive treatment with SiO₂ particles and threefold after 13 and 19 days post treatment.

The results show that administration of SiO₂ products of the QD class (ADAM-QD/S) regardless of invasive or non-invasive application helps alleviation of the symptoms of osteoarthritis and hence reduces pain upon walking. It is postulated that the mechanism involves eliminating/preventing the formation of underoxidized metabolic byproducts via processes by analogous modulation of redox catalysis as shown in Example 5.

6.4. IN VIVO Studies Relating to Osteoarthritic Changes with the Use of TiO₂ Crystals of the New QD Class (ADAM-QD/T)

The effects of TiO₂ crystals on osteoarthritic changes induced by unilateral surgical medial meniscal tear and medial collateral ligament transection in rats.

The objective of this study is to investigate the impact of TiO₂ crystals on osteoarthritic pain and histological changes in knee joints exhibiting unilateral medial meniscal tear and medial collateral ligament transection in young adult male rats.

Each group contained 12 male Lewis rats that were three months of age at the beginning of in-life phase of the study. Before the in-life phase, the rats were trained for pain measurement devices (hind paw weight distribution and paw withdrawal threshold.

Test item in the form of TiO₂ suspension was administered into the tight muscle (musculus quadriceps femoris) of the right hind limb (operated); Vehicle solution was administered into the same area of the non-operated left hind leg to avoid treatment-induced differences in pain measurement between the legs.

The test compound dosing suspension was 0.56 mg/ml, and dosed intramuscularly in thigh muscle of the right leg as 0.045 mg/kg and dosing volume 0.080 ml/kg.

Instrument of analysis: CatWalk XT computer-assisted method of locomotor analysis (Noldus Information Technology, Wageningen, The Netherlands).

The treatment with TiO₂ product significantly increased (by 13% difference in group average) weight bearing of the right hind limb, as well the guarding index (difference between weight bearing of the left heathy and right operated hind limb) compared to the vehicle-treated group on study day 19. In accordance there was a statistical trend of the weight bearing of the left hind limb being lower in the TiO₂ product group compared to the vehicle group similarly on study day 19.

Compared to the vehicle control, the pain tolerability threshold increased by 20% after 7 day (post treatment with TiO₂ product of the QD class (ADAM-QD/S)), whereas by 30% on day 13 and by 40% on the 19th day post treatment.

The results show that administration of nanoparticulate TiO₂ helps alleviation of the symptoms of osteoarthritis and hence reduces pain upon walking.

The results show that administration of TiO₂ products helps alleviation of the symptoms of osteoarthritis and hence reduces pain upon walking. Mechanism of TiO₂ products of the QD class involves eliminating and preventing the formation of underoxidized metabolic byproducts via processes by analogous modulation of redox catalysis as shown in Example 5.

ON THE FOLLOWING EXAMPLES OF EFFECTIVE IMPACT NOT ONLY ON THE DISEASE (PATHOLOGY) EXAMPLE, WHICH ILLUSTRATES A WIDE RANGE OF PROVIDING ANTIVIRAL EFFECT OF THE MEDICINAL PRODUCT FOR DIFFERENT PATHOGENS.

### Example 7

7.1. In Vitro Studies Related to SARS-COV2 Infections with the Use of SiO₂ Particles of the New QD Class (Adam-QD/S)

Testing the antiviral efficacy of SiO₂ particles against SARS-COV-2 in vitro Study number: 3025

A potential antiviral drug, SiO₂ suspension of the QD class was evaluated in vitro for its antiviral property against SARS-COV-2.

To test for antiviral activity of SiO₂ particles, virus infection and replication after the presence of the compound was evaluated. Before performing the above-mentioned assay, SARS-COV-2 was titrated on the Vero TMPRSS2 cells to determine the relevant titre. The compound was freshly diluted before each experiment in culture medium. To assess the potential antiviral activity against SARS-COV-2, the compound in six concentrations was added to VERO TMPRSS2 cells for 3 hrs. Hereafter, the SARS-COV-2 in three concentrations were added to the cells.

Representative SiO₂ suspensions of the QD class characterized by volume and surface weighted distribution per Example 4 were used for the studies.

Experimental set 1: After two days, cell monolayers were washed and stained for the presence of SARS-COV-2 antigen with an immunoperoxidase monolayer assay. Plates were read microscopically and judged for the presence of virus and immunostaining was scored in a semi-quantitative way: −: no cells; +: ≤25 cells; ++: >25 cells. In addition, spots in the individual wells were counted using an AID ELISPOT reader.

Experimental set 2: After two days, cells and supernatants were harvested and analysed for (the presence of cytotoxicity by LDH release.

SARS-COV-2 reproduction in VERO TMPRSS2 cells was as expected in the non-treated groups, whereas hIFNα was able to inhibit viral reproduction at the concentrations of 750 and 1000 U/ml at all three SARS-COV-2 concentrations used. The NIBSC reference serum inhibited SARS-COV-2 reproduction in a dose dependent manner at Multiplicity of infection of 0.005.

SiO₂ particles at concentrations of 50 μg/ml and 500 μg/ml inhibited the multiplication of SARS-COV-2, indicating that the compound has antiviral properties against SARS-COV-2.

The LDH cytotoxicity test showed a slight increase of the LDH activity in the supernatant of the SiO₂ particles treated group at a concentration of 500 μg/ml. This finding can be evaluated as a slight cytotoxicity effect.

The compound SiO₂ particles at concentrations 50 μg/ml and 500 μg/ml showed the inhibition of the reproduction of SARS-COV-2 (by reducing the mean SARS-CoV2-positive spots per M96-well from 600 to 230 and 130, respectively) which explicitly show that the compound has an antiviral property against SARS-COV-2.

THE FOLLOWING EXAMPLES ILLUSTRATE PROVIDING INDUCTION OF AN IMMUNE RESPONSE IN VERTEBRATES BY PHYSICAL OR CHEMICAL INTERACTIONS WITH ANTIGENS.

7.2. In Vivo Studies Related to SARS-Cov2 Infections with the Use of TiO₂ Crystals and SiO₂ Particles of the New QD Class (Adam-QD)

### Study Number: 23201

The aim of the study was to evaluate the adjuvant capacity of TiO₂ crystals and SiO₂ particles of the QD class using ovalbumin as a prototypical antigen and to compare them with a formulation consisting of an equivalent formulation of alum (Hydroxygel). This proof of concept study was aimed to study the capacity of the formulations to induce an antibody response, assess the immunization regimen for adverse effects on the animals (assessed by changes in weight) and to determine the predominant isotypes induced by each formulation.

### Methods:

Representative TiO₂ and SiO₂ products characterized by volume and surface weighted distribution per Example 4 was used for the studies.

Eight-week-old C57Bl/6 (B6) mice were immunized and received a boost under the same conditions in the same month.

The groups of mice were (8 mice per group):
1. Ovalbumin (10 μg)+phosphate buffered saline (PBS)
2. Ovalbumin (10 μg)+alum (1:1)
3. Ovalbumin (10 μg)+QD product TiO₂ (1:1)
4. Ovalbumin (10 μg)+QD product SiO₂ (1:1)

The mice were weighted at days 0, 1, 2, 3 and 14, 15, 16, 17, 18. The overall condition of the mice was checked visually every day, including weekends.

Antibody titers were measured by capture ELISA. Anti-ovalbumin IgM was measured in d0, d7, d14 and d28 sera. Total anti-ovalbumin IgG was measured in d0, d14 and d28 sera. Anti-ovalbumin IgG1, IgG2b, IgG2c and IgG3 were measured in d28 sera. None of the immunizations resulted in major changes in the weight of the mice. Evaluation of the adjuvant capacity of both of TiO₂ or SiO₂ QD products showed that both are capable of inducing a response similar to that demonstrated for the alum preparation. Among the differences observed between TiO₂ and SiO₂ products, the rapid induction of IgM response seems to indicate a stronger ability to activate B cells for the latter, which corresponds to a slight increase in the IgG3 isotype, which is the earliest, T-independent isotype exhibited in the B-cell response.

Overall, this study clearly demonstrates the adjuvant activity of TiO₂ and SiO₂ products against the prototypical antigen, ovalbumin, when using a specific formulation (1:1) and immunization route (intramuscular) increasing IgG titres fourfold at the end of the study compared to the control measurement.

THE FOLLOWING EXAMPLES ILLUSTRATE THE EFFECT OF THE QD PRODUCTS ON LIFESPAN.

### Example 8

### In Vivo Studies Related to Lifespan

### Autophagy Induction—Study No.: 2023794

Autophagy is essential for maintaining healthy cellular functioning and exhibits a gradual decrease in its capacity during aging which is associated with the incidence of various age-associated degenerative diseases, such cancer, neurodegeneration, tissue atrophy and fibrosis, is linked to defective autophagy. Abnormal cellular metabolism by aging is associated with the formation of unwanted byproducts leading to accumulation. The metabolic byproduct levels are higher at the end of lifespan and also when the oxidative homeostasis becomes unbalanced.

A tractable model tissue for studying the activity of autophagy is the fat body of the L3 stage Drosophila larva. In this organ at this stage, the basal level of autophagy is almost at an undetectable level. When a compound or environmental factor induces the process, fat body cells display an obvious amount of autophagic structures, autophagosomes and autolysosomes.

After 3 hours of treating the animals by a SiO₂ product of the QD class, fat bodies were isolated from 5 larvae, then examined by a Zeiss AxioImager M2 epifluorescent microscope equipped with a ApoTome 2 semi-confocal setup. Magnification 400× was used. For preparing pictures, the program Zeiss AxioVison 4.82 was applied. Two different reporters were used, GFP-p62/Ref (2) P and 3×mCherry-Atg8a. p62/Ref (2) P is a substrate of autophagy, so its level inversely correlates with the capacity of the process, whereas Atg8a serves as a key marker of autophagy which labels the membrane of early and late autophagic structures, autophagosomes and autolysosomes, respectively.

SiO₂ products of the QD class were tested on a model obtained from the fat body of L3 stage larvae of Drosophila melanogaster (fruit fly—a popular in vivo model for autophagy research), after the larvae were fed on a special culture medium containing nanoparticulate SiO₂ based on the developed methodology.

Using 3×mCherry-Atg8a marker, SiO₂ product showed an autophagy-inducing effect at a concentration of 1.2 mg/ml and at a concentration of 0.6 mg/ml in a concentration-dependent manner. Atg8a marker-positive autophagy structures were detected as red dots in Drosophila larval fat body. Nuclei were labeled with Hotchst dye (blue). The active substance of the QD class SiO₂ (ADAM-QD/S) significantly increases the amount of autophagic structures. The control value 2±1 (area ratio of 3×mCherry-Atg8a-positive structures) increased to 4±1 and 7±2 by use of QD products SiO₂ in 0.6 mg/ml concentration and in 1.2 mg/ml concentration, respectively.

The above results indicate that SiO₂ of the QD class has a significant potential in medical application as a potent autophagy-inducing agent which may find its use as a therapeutic substance to alleviate symptoms of age-related degenerative illnesses. Analogously to the previous examples, it is a well-founded explanation to the found effect that the mechanism involves eliminating and, importantly, preventing the formation of underoxidized metabolic byproducts via processes by analogous modulation of redox catalysis as shown in Example 5.

### Example 9

In Vitro Studies on Tumour Cell Lines: Antitumour Effects with the Use of TiO₂ Crystals of the New QD Class (ADAM-QD/T)

### 9.1. In Vitro Studies on Tumor Cell Lines:

1. Estimation on changes of cell viability the proportion of apoptotic and necrotic cells in different cell cultures:
HT-29 Human colon adenocarcinoma cell line.
ZR-75.1 Her-2 expressing human breast adenocarcinoma cell line.

In these studies cells (5×10⁴) were seeded in 6-well plates and cultured in RPMI-1640 medium containing 10% fetal bovine serum. The cultures incubated for 2 hours in CO₂ thermostate at 37° C. After the preincubation period the medium was removed, the cells were washed with physiological saline (2×1 ml 0.9% NaCl) and were exposed to TiO₂ suspension of the QD class at concentrations of 60 and 120 g/ml for 60 min. After removal of the test compounds, fresh RPMI 1640 medium containing 10% FBS was added and cultures were incubated for 24 h at 37° C. in a humidified thermostat (5% CO₂). At the end of the post-treatment period, the medium and culture supernatants containing released cells were removed and stored for cell counting. Cells in monolayer were released using trypsin EDTA. Cells obtained from both supernatants and monolayers were stained with propidium iodide and processed on a flow cytometer (FACS, Becton Dickinson, USA) Cells in the sub-G1 region were scored as apoptotic cells.

Results: In short-term studies (i.e., treatment before the onset of mitotic phase), the number of cells in TiO₂-treated cultures decreased slightly. At the same time, a large proportion of the cell population exhibited markers of apoptosis (10% in the control study, but 28% and more than 55% in the presence of TiO₂ of the QD class at concentrations of 60 and 120 μg/ml, respectively).

2. Measurement of cell kinetic parameters

To measure cell kinetic parameters, i.e. cells in DNA presynthetic phase (G1), DNA synthetic phase(S), premitotic or mitotic phase (G2/M), HT1080 fibrosarcoma cells were cultured as described above. After treatment for 48 hours, tumour cells were removed from the plates, stained with propidium iodide and cell cycle phases were analysed on a flow cytometer. (Becton-Dickinson USA).

Results: Treatment with the TiO₂ product of the QD class at a concentration of 120 μg/ml induced cell accumulation in the synthetic S-phase of DNA. Compared to the control value of 19.0%, this proportion increased to 26.8% in the presence of TiO₂ at a concentration of 120 μg/ml).

9.2. In Vivo Study of the Effect Against Human Tumours in Preclinical Models of HT-1080 Human Fibrosarcoma and ZR-75.1 Breast Adenocarcinoma with Transplantation into Nude Mice with the Use of TiO₂ Crystals of the New QD Class

### Experimental Animals:

C57Bl/6 male mice for transplanting Colon-38 and B-16 melanoma tumor as well as HT1080 human fibrosarcoma cell line and nude mice (SCID, C17/Icr, Charles River Laboratories, Germany) for studying human tumors were used.

To investigate the effect of TiO₂ crystals of the QD class against human tumors in preclinical models HT-1080 human fibrosarcoma and ZR-75.1 mammary adenocarcinoma were transplanted in nude mice. HT-1080 tumor cells (5×10⁴) were transplanted subcutaneously at the foot pad of the male nude mice. Upon reaching a palpable size (100 mg) treatment was started at 180 microgram nanosized TiO₂ dose, three times in every three days. ZR-75.1 mammary adenocarcinoma cells (5×10⁵) were transplanted in female nude mice at the mammary gland lines. Treatment was started when the tumor reached a palpable size, six times for three weeks. The change of the tumor volumes was determined as described above in case of murine tumors.

Results: The measurements of the life-span of the tumor bearing nude mice revealed a significant prolongation in the survival time:
37 days mean survival compared to 32 days recorded for the control group in the Colon-38 transplant group
28 days mean survival compared to 24 days recorded for the control group in the B-16 melanoma transplant group
25 days mean survival compared to 15 days recorded for the control group in the HT1080 human fibrosarcoma transplant group

The results of the in vivo experiments of Example 9.2. confirmed the in vitro data of Example 9.1. that the aqueous solution of TiO₂ crystals of the QD class has therapeutic potency in the test animals bearing human tumours.

### INDUSTRIAL APPLICABILITY

Used elaborated stable suspensions in composition of medical remedies have the following advantages:
It is catalyst of energy-exchange reactions of the ROS synthesis in the body;
Absence of general toxicity;
Absence of allergenic properties, i.e. hypoallergen;
Absence of harmful toxicological compounds;
Heterogenous suspension structures are photocatalysts having catalytic properties;
Have anti-pathogenous antiviral activity, preventing penetration of virus into the cells and promotes destruction of viruses with lipid membrane due to damaging of the latter.
It is possible to effectively influence onto pathogens.
Being a thermal catalyst for the synthesis of ROS, separately and/or in synergy It is possible to use high light powers (5 or more W/sq·cm.) It is possible to accelerate process of influence (up to 1-3 min).
Absence of toxic action of photocatalyst with/without photodynamic therapy (PDT);
Use of stable suspensions, obtained by the elaborated method, ensures antiviral therapeutic effect, antipathogen therapeutic effect, sorption capacity of crystals and particles ensures detoxication of organism, ensures elimination of underoxidation processes of in the body of living organism.
Provides the possibility of developing formulations with other active ingredients as well as forms of application (page 48, publication EP4272745A1).

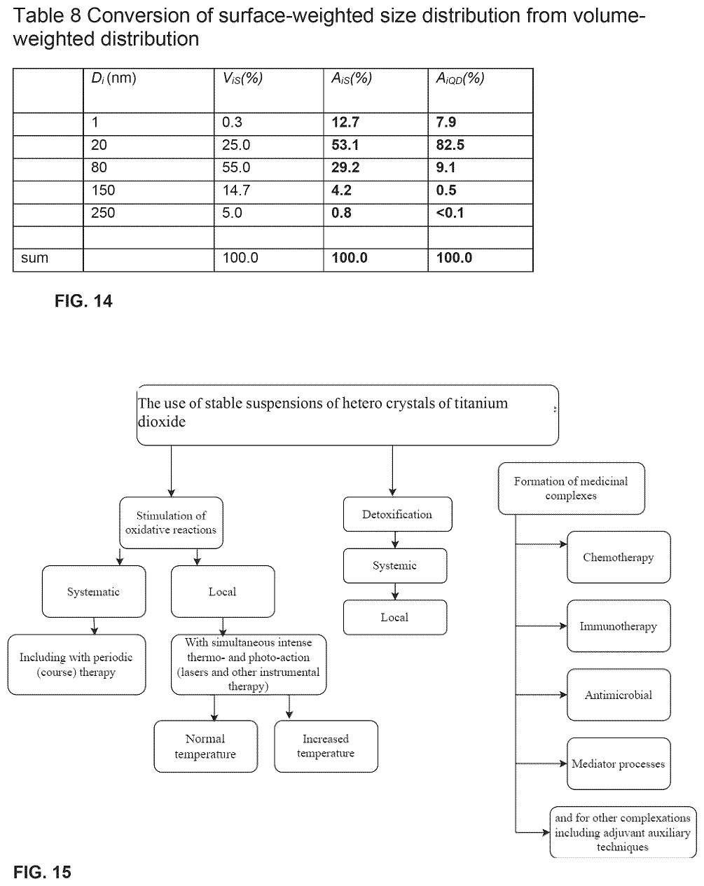

FIG. 15 shows use of suspensions in pharmaceutics for modern medicine.

1. The method of obtaining stable suspensions of heterocrystals of titanium dioxide or particles of silicon dioxide characterized by the fact that the starting material is in the form of aggregates with size more than 0.5 micrometer is mixed with an aqueous solution of pharmaceutically acceptable acid, with subsequent direction to homogenizing for the first stage of mixing, after that the obtained aqueous suspension is subjected to thermal treatment, then aqueous suspension is directed to the rotary rotor-type evaporator working under pressure lower than 100 kPa at temperature no more than 70° C. 70° C. for evaporation of pharmaceutically acceptable inorganic acid with suspension expense trough the rotor-type evaporator no more than 25 l/min and then the obtained activated particles are mixed with water in hydrodynamical cavitational homogenizer with regulated pulsating wave mode until obtaining stable suspension of heterocrystals of titanium dioxide or particles of silicon dioxide with size less than 450 nm, and presence on the lattice surface 60% up to 80% of electronically-excited triplet oxygen ³⁺TO₂3O₂ in the energy centers, namely, in the quantum dots—zones of local overheating, ensuring catalytic activity for formation of active forms of oxygen in the human body of a living organism.

2. The method of obtaining stable suspensions of heterocrystals of titanium dioxide or and particles of silicon dioxide according to the claim 1, characterized in the distinction it that the obtained aqueous suspension of heterocrystals of titanium dioxide or particles of silicon dioxide is periodically directed for thermal treatment to the ultrasonic bath with ultrasonic frequency 20-90 kHz and keep it at temperature no more than 70° C. during no more than 2 hours.

3. The method of obtaining stable suspensions of heterocrystals of titanium dioxide or and particles of silicon dioxide according to the claim 1, characterized in the distinction that the obtained activated particles TiO₂ or SiO₂ are mixed with water in the hydrodynamical cavitational homogenizer with regulated pulsating wave mode until obtaining stable suspension of heterocrystals of titanium dioxide or particles of silicon dioxide from 0.0001-10 mass % concentration in suspension condition.

4. The method of obtaining stable suspensions of heterocrystals of titanium dioxide or and particles of silicon dioxide according to the claim 1, characterized in the distinction is that in the cavitational homogenizer a flow of mixed medium passes through the first block of preliminary mixing and further through the block of cavitational homogenizing, and then through the block with possibility of regulation in a flow of the ingredient dosing with subsequent periodical direction into the block of regulated output of homogenized product subjected to pulsation-wave homogenizing, at that the pulsation chamber of wave mixing is executed with regulated reflector of flow mixture, installed at the chamber output.

5. The method of obtaining stable suspensions of heterocrystals of titanium dioxide or and particles of silicon dioxide according to the claim 1, characterized in the distinction that activated crystals TiO₂ or particles SiO₂ are obtained with presence in structure of oxygen from 60% up to 80% in metastable electronically-excited third state triplet condition ³⁺TO₂3O₂, at that the particles TiO₂ have Zeta-potential +30-+15 mV, and the particles SiO₂ have Zeta-potential −20-−15 mV and are characterized with presence of sorption properties.

6. Stable suspension of heterocrystals of titanium dioxide and particles of silicon dioxide obtained according to the claim 1 characterized by distribution of activated crystals of titanium dioxide with size up to 1 nm being 0.3 vol %, up to 20 nm being 5-40 vol %, particles with size up to 80 nm being 10-80 vol %, particles with size up to 150 nm being 5-30 vol %, particles with size up to 250 nm being 5-20 vol %, particles with size more than 250 nm-no more than 10 vol %, and distribution of activated particles of silicon dioxide with size 40-80 nm being 10-80 vol %, particles with size 80-150 nm being 10-80 vol %, particles with size 150-250 nm being less than 30 vol %, particles with size more than 250-450 nm-no more than 15%, and with presence on the lattice surface from 60% of up to 80% of electronically-excited third state oxygen ³⁺TO₂3O2 in the energy centers, namely, in the quantum dots-zones of local overheating, ensuring catalytic activity for formation of Active Forms of Oxygen in the human body of living organism.

7. Use of stable suspensions of heterocrystals of titanium dioxide or and particles of silicon dioxide obtained according to the claim 6 in medical agents and in combination with other active pharmaceutical components, intravenously, intramuscularly, orally, nasally, vaginally, rectally, locally (ENT) use or topically, at that therapeutically important amount of an active substance of crystals or particles is for use: intravenous from 0.006 mg/ml, intramuscular from 0.01 mg/ml, subcutaneous from 0.1 mg/ml, orally from 0.01 mg/ml, nasally from 0.01 mg/ml, vaginally from 0.01 mg/ml, rectally from 0.01 mg/ml, local ENT use from 0.01 mg/ml, ocular use from 0.001 mg/ml, topical from 0.0003 mg/ml, as well as mucous, membranes, subcutaneous, intravascular, capillary, droplet, transmembrane and dermal, intracellular injection including locally into cell structures.

8. Use of stable suspensions of heterocrystals of titanium dioxide and particles of silicon dioxide according to the claim 6 as thermocatalystscatalysts of conversion of oxygen from triplet ³⁺TO₂³O₂ into singlet ¹⁻³SO₂ condition O2S1-3 condition at the expense of energy of thermal energy, including physiological temperature of from 36.6° C. in the human body and/or photonic hyperthermia and/or wave, and electromagnetic and resonant radiation, ensuring energy needed for conversion of oxygen from triplet into singlet condition for synthesis of active forms of oxygen in the human body.

9. Use of stable suspensions of heterocrystals of titanium dioxide and particles of silicon dioxide according to the claim 6, the distinction is that for induction of formation of active forms of oxygen in the inflammation focuses in presence of NAD+ enzyme of immune cells target delivery of said stable suspension is ensured into the areas of inflammation localization.

10. Use of stable suspensions of heterocrystals of titanium dioxide and particles of silicon dioxide according to the claim 6, characterized by the fact that antiviral effect of a medical agent is ensured.

11. Use of stable suspensions of heterocrystals of titanium dioxide and particles of silicon dioxide according to the claim 6, the distinction is that antipathogenous antiviral effect of a medical agent is ensured.

12. Use of stable suspensions of heterocrystals of titanium dioxide and particles of silicon dioxide according to the claim 6, the distinction is that sorption ability of the crystals and particles in a medical agent ensures detoxication of organism.

13. Use of stable suspensions of heterocrystals of titanium dioxide and particles of silicon dioxide according to the claim 6, the distinction is that it ensures in a medical agent elimination of under oxidation processes in the human body of living organism.

14. Use of stable suspensions of heterocrystals of titanium dioxide and particles of silicon dioxide according to the claim 6, the distinction is that induction of immune response of a vertebrate vertebrata by means of physical or chemical interaction with antigens is ensured.

15. The method of obtaining stable suspensions of heterocrystals of titanium dioxide or and particles of silicon dioxide according to the claim 2, characterized in the distinction is that in the cavitational homogenizer a flow of mixed medium passes through the first block of preliminary mixing and further through the block of cavitational homogenizing, and then through the block with possibility of regulation in a flow of the ingredient dosing with subsequent periodical direction into the block of regulated output of homogenized product subjected to pulsation-wave homogenizing, at that the pulsation chamber of wave mixing is executed with regulated reflector of flow mixture, installed at the chamber output.

16. The method of obtaining stable suspensions of heterocrystals of titanium dioxide or and particles of silicon dioxide according to the claim 3, characterized in the distinction is that in the cavitational homogenizer a flow of mixed medium passes through the first block of preliminary mixing and further through the block of cavitational homogenizing, and then through the block with possibility of regulation in a flow of the ingredient dosing with subsequent periodical direction into the block of regulated output of homogenized product subjected to pulsation-wave homogenizing, at that the pulsation chamber of wave mixing is executed with regulated reflector of flow mixture, installed at the chamber output.

17. The method of obtaining stable suspensions of heterocrystals of titanium dioxide or and particles of silicon dioxide according to the claim 4, characterized in the distinction that activated crystals TiO₂ or particles SiO₂ are obtained with presence in structure of oxygen from 60% up to 80% in metastable electronically-excited third state triplet condition ³⁺TO₂3O₂, at that the particles TiO₂ have Zeta-potential +30-+15 mV, and the particles SiO₂ have Zeta-potential −20-−15 mV and are characterized with presence of sorption properties.
